(19) United States
(12) Patent Application Publication
(10) Pub. No.: US 2021/0295954 A1
(43) Pub. Date: Sep. 23, 2021

(54) SOFTWARE-BASED ECOSYSTEM FOR USE WITH A RAPID TEST

(71) Applicant: Detect, Inc., Guilford, CT (US)

(72) Inventors: Jonathan M. Rothberg, Guilford, CT (US); Benjamin Rosenbluth, Hamden, CT (US)

(73) Assignee: Detect, Inc., Guilford, CT (US)

(21) Appl. No.: 17/203,446

(22) Filed: Mar. 16, 2021

Related U.S. Application Data
(60) Provisional application No. 63/116,603, filed on Nov. 20, 2020.
(60) Provisional application No. 63/081,201, filed on Sep. 21, 2020.
(60) Provisional application No. 63/074,524, filed on Sep. 4, 2020.
(60) Provisional application No. 63/068,303, filed on Aug. 20, 2020.
(60) Provisional application No. 63/065,131, filed on Aug. 13, 2020.
(60) Provisional application No. 63/059,928, filed on Jul. 31, 2020.
(60) Provisional application No. 63/053,534, filed on Jul. 17, 2020.
(60) Provisional application No. 63/036,887, filed on Jun. 9, 2020.
(60) Provisional application No. 63/027,859, filed on May 20, 2020.
(60) Provisional application No. 63/027,874, filed on May 20, 2020.
(60) Provisional application No. 63/027,890, filed on May 20, 2020.
(60) Provisional application No. 63/027,864, filed on May 20, 2020.
(60) Provisional application No. 63/027,878, filed on May 20, 2020.
(60) Provisional application No. 63/027,886, filed on May 20, 2020.
(60) Provisional application No. 63/022,534, filed on May 10, 2020.
(60) Provisional application No. 63/022,533, filed on May 10, 2020.
(60) Provisional application No. 63/016,797, filed on Apr. 28, 2020.
(60) Provisional application No. 63/013,450, filed on Apr. 21, 2020.
(60) Provisional application No. 63/010,578, filed on Apr. 15, 2020.
(60) Provisional application No. 63/010,626, filed on Apr. 15, 2020.
(60) Provisional application No. 63/002,209, filed on Mar. 30, 2020.
(60) Provisional application No. 62/991,039, filed on Mar. 17, 2020.

(51) Int. Cl. G16B 50/30; G16H 10/40; G16H 50/80; G16H 10/60

(52) U.S. Cl. CPC G16B 50/30; G16H 10/60; G16H 50/80; G16H 10/40

(57) ABSTRACT

Described herein in one embodiment is software, which may be downloaded to a device, to guide a user through administration of a test. Test results may be uploaded, manually or automatically, to a device or communicated remotely through a network. In an embodiment, the test is a rapid test. In an embodiment, the rapid test detects presence of COVID-19. In an embodiment, the rapid test detects COVID-19 or influenza. In an embodiment, the rapid test detects influenza A or influenza B. In an embodiment, the rapid test detects a target nucleic acid. In an embodiment, the target nucleic acid represents one of a viral, bacterial, fungal, parasitic or protozoan pathogen. Each of the rapid tests may be self administrable.

### RELATED APPLICATIONS

This application claims priority under 35 U.S.C. § 119(e) to U.S. Provisional Patent Application No. 62/991,039, titled “VIRAL RAPID TEST,” filed Mar. 17, 2020; U.S. Provisional Patent Application No. 63/002,209, titled “VIRAL RAPID TEST,” filed Mar. 30, 2020; U.S. Provisional Patent Application No. 63/010,578, titled “VIRAL RAPID TEST,” filed Apr. 15, 2020; U.S. Provisional Patent Application No. 63/010,626, titled “VIRAL RAPID COLORIMETRIC TEST,” filed Apr. 15, 2020; U.S. Provisional Patent Application No. 63/013,450, titled “METHOD OF MAKING AND USING A VIRAL TEST KIT,” filed Apr. 21, 2020; U.S. Provisional Patent Application No. 63/016,797, titled “SAMPLE SWAB WITH BUILD-IN ILLNESS TEST,” filed Apr. 28, 2020; U.S. Provisional Patent Application No. 63/022,534, titled “RAPID DIAGNOSTIC TEST,” filed May 10, 2020; U.S. Provisional Patent Application No. 63/022,533, titled “RAPID DIAGNOSTIC TEST,” filed May 10, 2020; U.S. Provisional Patent Application No. 63/036,887, titled “RAPID DIAGNOSTIC TEST,” filed Jun. 9, 2020; U.S. Provisional Patent Application No. 63/074,524, titled “RAPID DIAGNOSTIC TEST WITH INTEGRATED SWAB,” filed Sep. 4, 2020; U.S. Provisional Patent Application No. 63/081,201, titled “RAPID DIAGNOSTIC TEST,” filed Sep. 21, 2020; U.S. Provisional Patent Application No. 63/065,131, titled “APPARATUSES AND METHODS FOR PERFORMING RAPID DIAGNOSTIC TESTS,” filed Aug. 13, 2020; U.S. Provisional Patent Application No. 63/059,928, titled “RAPID DIAGNOSTIC TEST,” filed Jul. 31, 2020; U.S. Provisional Patent Application No. 63/068,303, titled “APPARATUSES AND METHODS FOR PERFORMING RAPID MULTIPLEXED DIAGNOSTIC TESTS,” filed Aug. 20, 2020; U.S. Provisional Patent Application No. 63/027,859, titled “RAPID SELF ADMINISTRABLE TEST,” filed May 20, 2020; U.S. Provisional Patent Application No. 63/027,874, titled “RAPID SELF ADMINISTRABLE TEST,” filed May 20, 2020; U.S. Provisional Patent Application No. 63/027,890, titled “RAPID SELF ADMINISTRABLE TEST,” filed May 20, 2020; U.S. Provisional Patent Application No. 63/027,864, titled “RAPID SELF ADMINISTRABLE TEST,” filed on May 20, 2020; U.S. Provisional Patent Application No. 63/027,878, titled “RAPID SELF ADMINISTRABLE TEST,” filed on May 20, 2020; U.S. Provisional Patent Application No. 63/027,886, titled “RAPID SELF ADMINISTRABLE TEST,” filed May 20, 2020; U.S. Provisional Patent Application No. 63/053,534, titled “COMPUTER VISION ALGORITHM FOR DIAGNOSTIC TESTING,” filed Jul. 17, 2020; and U.S. Provisional Patent Application No. 63/116,603, titled “SOFTWARE ECOSYSTEM FOR HEALTH MONITORING,” filed Nov. 20, 2020, each of which is hereby incorporated by reference herein in its entirety.

### BACKGROUND

Rapid disease detection methods have been developed to provide diagnostic answers at the point-of-care without complex lab equipment. Viral infections, such as coronaviruses and influenzas, commonly cause respiratory tract infections in humans. While in some humans, such viral infections are mild to moderate, in others the infections are severe and even fatal. Certain viruses, such as the novel coronavirus disease 2019 (COVID-19), have proven to be more fatal than other viral infections. The current lack of treatment or vaccine for this novel virus has resulted in a pandemic. The ongoing crisis associated with COVID-19 illustrates the importance of developing rapid disease testing methods, so that populations may be tested more efficiently and appropriate public health measures may be enacted.

### SUMMARY

In an embodiment, a software-based testing ecosystem is provided for receiving health information about a patient, including disease or antibody test data. The testing ecosystem can integrate various data to provide a central medical and testing resource for users to track disease progression. The testing ecosystem can integrate data provided by users and/or data that is obtainable from other resources. In some embodiments, the data integrated into the ecosystem includes user information, account information, medical records, rapid test data, other testing data (e.g., antibody tests and/or other viral, bacterial, fungal, parasitic and/or protozoan pathogen tests), and/or the like. In some embodiments, the patient information includes at least one of name, social security number, date of birth, address, phone number, email address, medical history, and medications. In an embodiment, the data can be obtained from resources such as clinician databases, medical record databases, agency databases, and/or any other resources with relevant data.

In an embodiment, the software-based ecosystem integrates a reading and/or result of a rapid test that detects the presence of COVID-19 and/or influenza and/or a target nucleic acid. Influenza may include influenza A or influenza B. In an embodiment, the target nucleic acid represents one of a viral, bacterial, fungal, parasitic or protozoan pathogen. In some embodiments, such a rapid test may be self-administrable.

In an embodiment, the software ecosystem receives and/or processes results of an antibody or antigen test. A test reading, such as a test reading displayed on a strip, may be read or uploadable to a smart device or communicated through a network to the ecosystem. The reading may be entered by a user manually or an image of the reading may be uploaded. In an embodiment, a software application is configured to interact with the testing ecosystem. The software application can upload a test reading, a test result and/or other subject information to the testing ecosystem. In an embodiment, the software ecosystem automatically analyzes the reading and provides a positive or negative test result.

In an embodiment, the software-based testing ecosystem includes one more compute resources and/or databases to store test results and patient information. The testing ecosystem can store the information in a central database and/or send the information to one or more other locations (e.g., clinicians, authorities, etc.). In an embodiment, the software application sends test information (e.g., test readings and/or information) to a secure, HIPAA-compliant, cloud-based software infrastructure of the software ecosystem. The software ecosystem can therefore facilitate simple, fast, and scalable reporting to the federal and state health agencies.

In an embodiment, the software-based ecosystem includes user or patient tracking capabilities, such as with use of smartphones or remote devices with tracking capabilities. The testing ecosystem can store tracking information from the subject and/or other users of the testing ecosystem, and use the tracking information to provide additional services, such as contact tracing. The locations may also be communicated to a central database server and/or to a remote doctor or other.

In an embodiment, a test ecosystem is provided that is configured to process a test reading or a test result of a rapid test for COVID-19 and/or an influenza virus and/or a target nucleic acid, wherein the rapid test is an isothermal nucleic acid amplification based test.

In an embodiment, the test ecosystem comprises a computing resource configured to store the test reading or the test result.

In an embodiment, the computing resource is a central computing resource, a clinician computing resource, a medical record computing resource, and/or an agency computing resource.

In an embodiment, the test ecosystem is configured to integrate the test reading or the test result with subject data.

In an embodiment, the subject data is account data, tracking data, test record data, and/or clinical data.

In an embodiment, the test ecosystem is configured to perform contact tracing based on the tracking data.

In an embodiment, the test record data comprises antibody test data, COVID-19 test data, influenza test data, and/or target nucleic acid test data.

In an embodiment, the testing ecosystem is configured to access at least a first portion of the subject data from the clinician computing resource, the medical record computing resource, and/or the agency computing resource.

In an embodiment, the test ecosystem is configured to store the test reading, the test result, and/or the subject data in the central computing resource.

In an embodiment, the testing ecosystem is configured to transmit the test reading, the test result, and/or at least a second portion of the subject data to the clinician computing resource, the medical record computing resource, and/or the agency computing resource.

In an embodiment, the test ecosystem is configured to process the test result of the rapid test for COVID-19.

In an embodiment, the test ecosystem is configured to process the test result of the rapid test for an influenza virus.

In an embodiment, the influenza virus is an influenza A virus or an influenza B virus. In an embodiment, the test ecosystem is configured to process the test result of the rapid test for the target nucleic acid.

In an embodiment, the target nucleic acid is any of a viral, bacterial, fungal, parasitic and/or protozoan pathogen.

In an embodiment, the rapid test is self administrable.

In an embodiment, a test reading of the rapid test may be entered manually by a user.

In an embodiment, the test reading may be entered by a user through uploading an image of the test reading using a downloadable software application.

In an embodiment, the test ecosystem determines the test result automatically from the entered test reading.

In an embodiment, the downloadable software application guides a user to perform the following steps: (a) collect a sample from a subject; and (b) add one or more reagents to the sample.

In an embodiment, an apparatus is provided comprising: at least one computer hardware processor; and at least one non-transitory computer-readable storage medium storing processor executable instructions that, when executed by the at least one computer hardware processor, cause the at least one computer hardware processor to perform: processing a reading of a rapid test for COVID-19 and/or an influenza virus and/or a target nucleic acid, wherein the rapid test is an isothermal nucleic acid amplification based test.

In an embodiment, the instructions are configured to cause the at least one computer hardware processor to receive the reading of the rapid test for COVID-19.

In an embodiment, the instructions are configured to cause the at least one computer hardware processor to receive the reading of the rapid test for an influenza virus.

In an embodiment, the influenza virus is an influenza A virus or an influenza B virus. In an embodiment, the instructions are configured to cause the at least one computer hardware processor to receive the reading of the rapid test for the target nucleic acid.

In an embodiment, the target nucleic acid is any of a viral, bacterial, fungal, parasitic and/or protozoan pathogen.

In an embodiment, the rapid test is self administrable.

In an embodiment, the instructions are configured to cause the at least one computer hardware processor to receive manual entry of the test reading by a user.

In an embodiment, the instructions are configured to cause the at least one computer hardware processor to receive an uploaded image of the test reading.

In an embodiment, the instructions are configured to cause the at least one computer hardware processor to provide a test result automatically from the entered test reading.

In an embodiment, the apparatus comprises a computing resource configured to store the test reading or the test result.

In an embodiment, the computing resource is a central computing resource, a clinician computing resource, a medical record computing resource, and/or an agency computing resource.

In an embodiment, the instructions are configured to cause the at least one computer hardware processor to integrate the test reading or the test result with subject data.

In an embodiment, the subject data is account data, tracking data, test record data, and/or clinical data.

In an embodiment, the instructions are configured to cause the at least one processor to perform contact tracing based on the tracking data.

In an embodiment, a computerized method is provided comprising: processing a reading of a rapid test for COVID-19 and/or an influenza virus and/or a target nucleic acid, wherein the rapid test is an isothermal nucleic acid amplification based test.

In an embodiment, the method includes receiving the reading of the rapid test for COVID-19.

In an embodiment, the method includes receiving the reading of the rapid test for an influenza virus.

In an embodiment, the influenza virus is an influenza A virus or an influenza B virus. In an embodiment, the method includes receiving the reading of the rapid test for the target nucleic acid.

In an embodiment, the target nucleic acid is any of a viral, bacterial, fungal, parasitic and/or protozoan pathogen.

In an embodiment, the rapid test is self administrable.

In an embodiment, the method further includes receiving manual entry of the test reading by a user.

In an embodiment, the method further includes receiving an uploaded image of the test reading.

In an embodiment, the method further includes providing a test result automatically from the entered test reading.

In an embodiment, the method further includes receiving the reading from a downloaded software application.

In an embodiment, the method further includes storing the test reading or the test result in a computing resource.

In an embodiment, the computing resource is a central computing resource, a clinician computing resource, a medical record computing resource, and/or an agency computing resource.

In an embodiment, the method further includes integrating the test reading or the test result with subject data.

In an embodiment, the subject data is account data, tracking data, test record data, and/or clinical data.

In an embodiment, the instructions are configured to cause the at least one processor to perform contact tracing based on the tracking data.

### BRIEF DESCRIPTION OF THE DRAWINGS

FIG. 1 shows an exemplary computerized method for guiding a user through the steps to use a test, according to some embodiments.

FIGS. 2A-2F show an exemplary sequence of steps for administering a diagnostic test, according to some embodiments.

FIGS. 3A-3L are, according to some embodiments, schematic illustrations of steps of a diagnostic testing method.

FIG. 4 is an exemplary computerized method for receiving and/or processing a test reading and/or result, according to some embodiments.

FIG. 5 illustrates an exemplary method for determining diagnostic test results, according to some embodiments.

FIGS. 6A-6B show screenshots from the companion mobile application (app), including the “Record Results” screen, “Image Acquisition” screen, “Test Complete” screen for negative test result, and “Test Complete” screen for positive test result.

FIG. 7 shows an exemplary lateral flow strip key for interpreting results, including the key itself, a positive test, and a negative test.

FIG. 8A is a diagram of exemplary components of a testing ecosystem, according to some embodiments.

FIG. 8B is a diagram showing examples of various data that can be integrated into a testing ecosystem, according to some embodiments.

FIG. 9 is a diagram of an exemplary computerized method for configuring user-specific data and integrating a test result with the testing ecosystem, according to some embodiments.

FIG. 10 is a flow chart of an exemplary computerized method for uploading test result data and/or other data to the ecosystem, according to some embodiments.

FIG. 11 is a flow chart of an exemplary computerized method for aggregating user information, according to some embodiments.

FIG. 12 is a flow chart of an exemplary computerized method for performing contact tracing, according to some embodiments.

FIG. 13 is a diagram illustrating detection of virus and antibodies over time.

FIG. 14 is a diagram illustrating a timeline of viral infection including viral and serological antibody testing.

FIG. 15 is a diagram illustrating an exemplary computer system that may be used to perform any of the aspects of the techniques and embodiments disclosed herein.

FIG. 16 is a schematic depicting an embodiment of a testing procedure described herein.

FIGS. 17A-17B are diagrams depicting an embodiment of a cartridge.

FIGS. 18A-18B are diagrams depicting an embodiment a cartridge.

FIG. 19 depicts a prototype of a cartridge embodiment described herein.

FIG. 20 depicts a test kit according to an embodiment.

FIG. 21 is a schematic illustration of an exemplary blister pack set up.

FIG. 22 illustrate three different blister pack configurations.

FIG. 23 is a schematic depicting an embodiment of the swabs described herein.

FIG. 24 shows an embodiment of a test kit with a built in sample collection swab.

FIG. 25 is a schematic illustration of a substrate of a diagnostic device, according to some embodiments.

FIGS. 26A-26C are, according to some embodiments, schematic illustrations of an inner component and a substrate of a diagnostic device.

FIG. 27 is a schematic illustration of an outer casing of a diagnostic device, according to some embodiments.

FIGS. 28A-28B are schematic illustrations of diagnostic testing kits, according to some embodiments.

### DETAILED DESCRIPTION

In an embodiment, envisioned is a downloadable software application to guide a user through use of a home test kit for testing for a viral illness such as COVID19, influenza type A and/or influenza type B, a software-based ecosystem for processing test results from the home test kit, and a communication protocol enabling communication of test results. In an embodiment, the software ecosystem can process test results from the home test kit and other tests, and integrate the test results with other patient information, including electronic health records, antibody tests, etc., which can be stored in the ecosystem, reported to clinicians, and/or the like.

I. Downloadable Software Application for Guiding a User Through Test Administration

In an embodiment, a software application can provide instructions to guide a user through performing a test. The instructions may include instructions for the use, assembly, and/or storage of the diagnostic device and/or any other components associated with the kit. By using the software application described herein, diagnostic devices, systems, and methods described herein may be safely and easily operated or conducted by untrained individuals (e.g., untrained clinicians, at-home users, etc.). Unlike prior art diagnostic tests, some embodiments described herein may not require knowledge of even basic laboratory techniques (e.g., pipetting). Further, due to the rapid spread and evolution diseases, it is desirable to quickly administer tests in a manner that does not necessarily require training to understand how to administer a test. Therefore, the software application described herein can be provided in conjunction with these and other tests in order to provide for rapid deployment and use of tests.

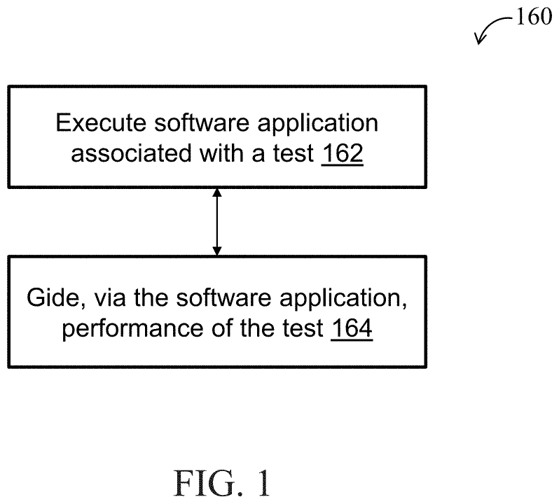

FIG. 1 shows an exemplary computerized method 160 that can be performed by the software application for guiding a user through the steps to use a test, according to some embodiments. At step 162, the device executes a software application associated with the test. In an embodiment, the device is a computer and/or a smart device. The smart device can be a smartphone, a smartwatch, and/or a smart home device. The test can be any of the exemplary tests and/or test steps described herein. In an embodiment, the software application can be downloaded to a device.

At step 164, the software application guides a user to perform the test. In an embodiment, the software application guides a user through administration of a test for diagnosing presence of COVID-19. In an embodiment, the software application guides a user through administration of a test for diagnosing presence of influenza. In an embodiment, the software application guides a user through administration of a test for diagnosing presence of influenza A or influenza B. In an embodiment, the software application guides a user through administration of a test for diagnosing presence of a target nucleic acid. In an embodiment, the target nucleic acid represents one of a viral, bacterial, fungal, parasitic or protozoan pathogen. The user, in some embodiments, is the person (or people) performing the test.

The software application can provide the instructions and/or step(s) using any form recognizable by one of ordinary skill in the art as a suitable vehicle for containing such instructions. In an embodiment, the software application uses audio, sensory, and/or visual techniques to guide a user through the test, including but not limited to user interfaces, images, sounds, lights, haptic feedback, and/or the like. For example, the instructions may be written or published, verbal, audible (e.g., telephonic), digital, optical, visual (e.g., videotape, DVD, etc.) or electronic communications (including Internet or web-based communications).

In an embodiment, the downloadable software application guides a user to collect a sample from a subject, and to add one or more reagents to the sample. In an embodiment, the downloadable software application guides the user to add the one or more reagents in a sequential order. For example, in certain instances, the instructions may instruct a user when to change reaction tube caps and how to release reagents from the reaction tube caps (e.g., by depressing a button, twisting a portion of the reaction tube cap, etc.). In an embodiment, the one or more reagents comprise one or more lysis reagents, RNA extraction reagents, and/or amplification reagents. In an embodiment, the amplification reagents comprise reverse transcriptase and/or a DNA-dependent polymerase. In an embodiment, the amplification reagents comprise one or more RPA reagents and/or one or more LAMP reagents. In an embodiment, at least one of the one or more reagents is lyophilized.

In some embodiments, the instructions instruct a user on beginning and/or ending heating protocols. In some cases, a user may receive an alert (e.g., on a mobile application) when a heating protocol (e.g., a lysis heating protocol, an amplification heating protocol) is complete. In some embodiments, the software-based application may be connected (e.g., via a wired or wireless connection) to one or more components of a diagnostic system. In certain embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

The software application can be used to validate one or more steps of the test process were performed correctly. In an embodiment, the downloadable software application confirms that the one or more reagents were added in a correct order. In an embodiment, the downloadable software application confirms that the one or more reagents were added at a correct time. In an embodiment, the downloadable software application uses a camera function to validate a color of a solution formed by adding the one or more reagents to the sample.

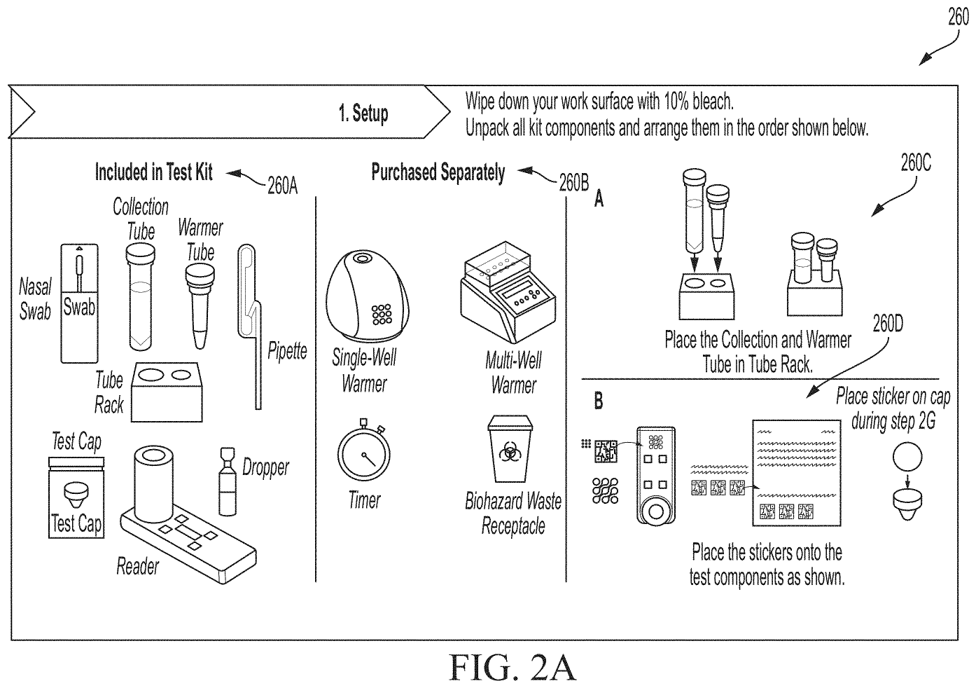

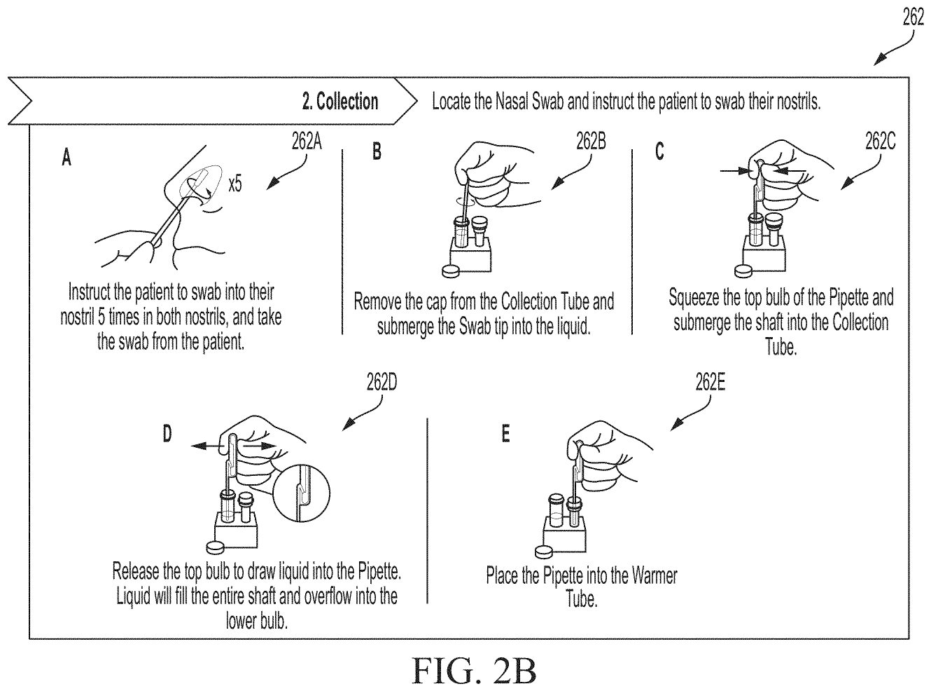

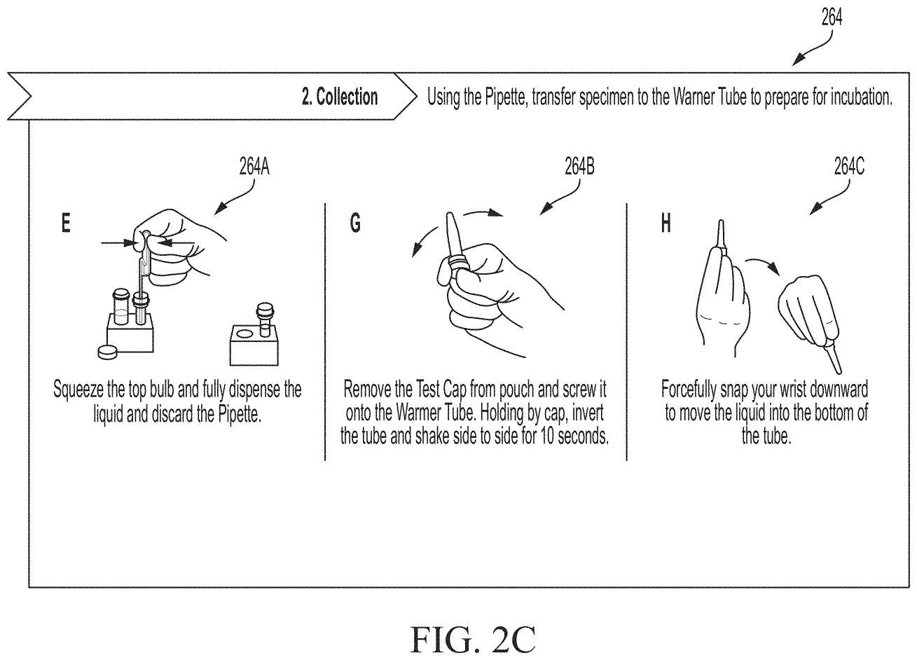

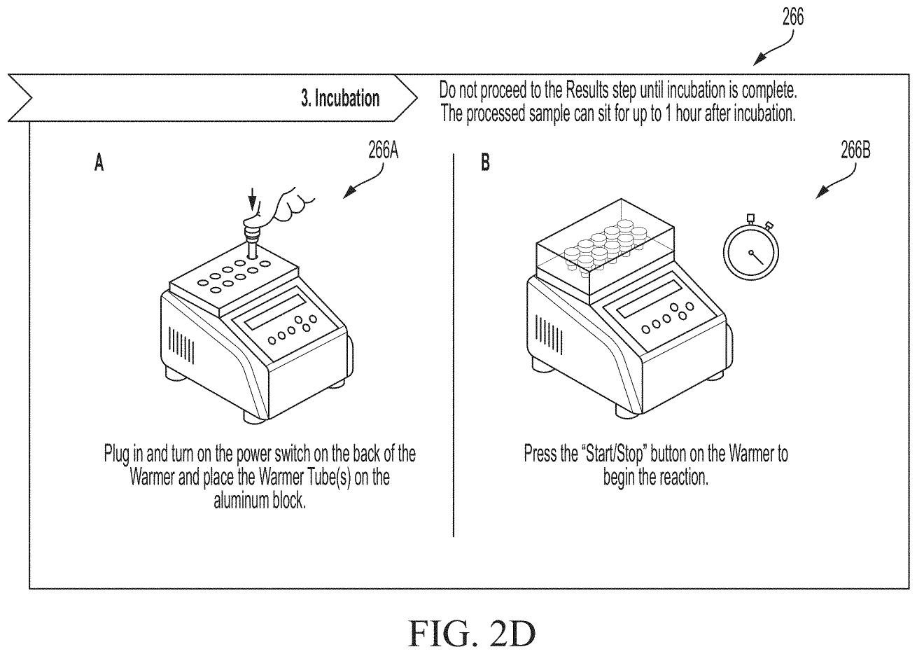

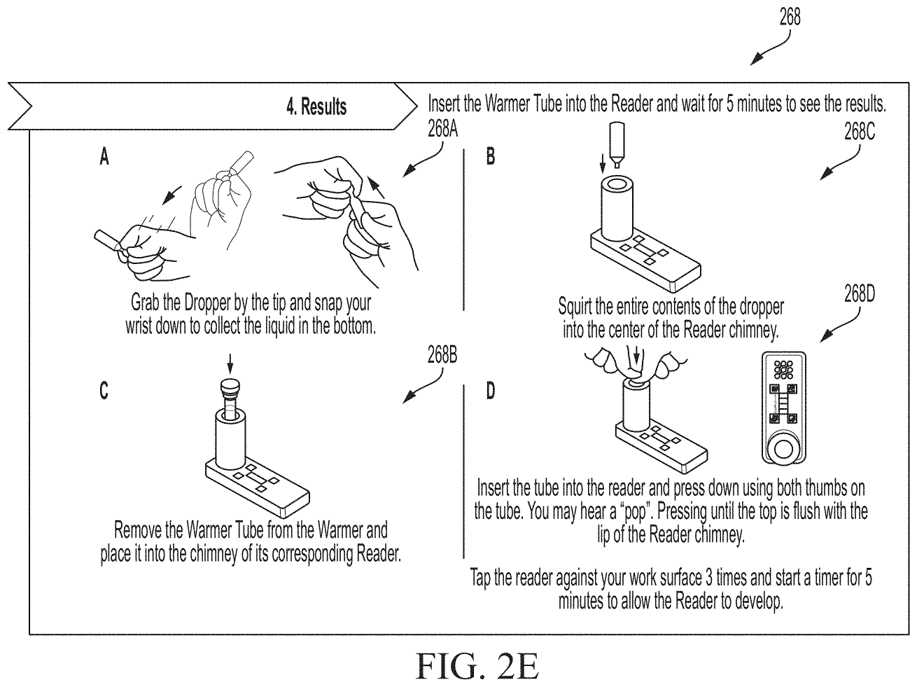

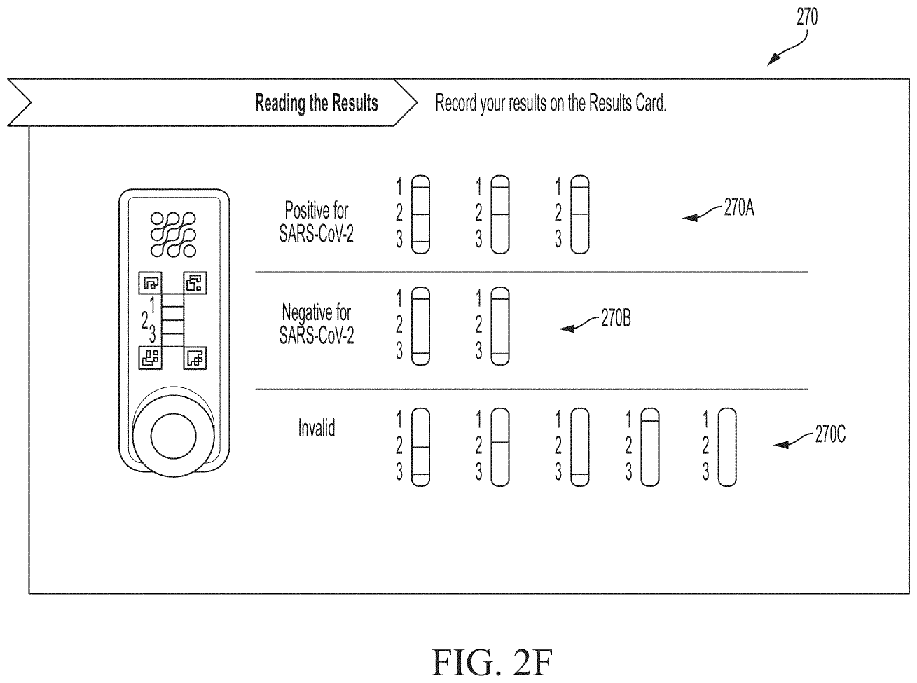

In an embodiment, the software application is configured to provide a series of guided steps to illustrate the sequence of steps. FIGS. 2A-2F show an exemplary sequence of steps for administering a diagnostic test, according to some embodiments. In an embodiment, the software application can present the guided steps using a series of user interfaces (e.g., including one or more interfaces, as desired, for each of the exemplary steps). In some embodiments, the software application can be configured to provide instructions using other techniques, such as verbal, audible, etc. as described herein. It should be appreciated that the sequence of steps shown in FIGS. 2A-2F are just one example of the many (and various) test sequences that a user can be guided through to perform a test.

FIG. 2A is an exemplary interface 260 for a test setup, according to one embodiment. The interface 260 includes a first portion 260A that shows the items in the test kit, a second portion 260B that shows separately-purchased items, a third portion 260C that instructs the user to place the collection and warmer tube in the tube rack, and a fourth portion 260D that instructs the user to place the stickers onto the test components. As explained herein, it should be appreciated that a plurality of interfaces can be used to illustrate the test steps (e.g., four different interfaces can be presented for each of the four portions 260A-260D of interface 260).

FIG. 2B is an exemplary interface 262 showing collection steps of a test, according to one embodiment. The interface 262 includes a first portion 262A that instructs the patient/user to swab into their nostril five (5) times in both nostrils, a second portion 262B that instructs the user to remove the cap from the collection tube and submerge the swab tip into the liquid, a third portion 262C that instructs the user to squeeze the top bulb of the pipette and submerge the shaft into the collection tube, a fourth portion 262D that instructs the user to release the top bulb to draw liquid into the pipette, such that liquid will fill the entire shaft and overflow into the lower bulb, and a fifth portion 262E that instructs the user to place the pipette into the warmer tube.

FIG. 2C is an exemplary interface 264 showing further collection steps of a test, according to one embodiment. The interface 264 includes a first portion 264A that instructs the user to squeeze the top bulb and fully dispense the liquid and discard the pipette, a second portion 264B that instructs the user to remove the test cap from the pouch and screw it onto the warmer tube, and holding the cap to invert the tube and shake it for ten (10 seconds, and a third portion 264C that instructs the user to forcefully snap their wrist downward to move the liquid into the bottom of the tube.

FIG. 2D is an exemplary interface 266 showing incubation steps of a test, according to one embodiment. The interface 266 includes a first portion 266A that instructs a user to turn on the warmer and place the warmer tubes on the aluminum block of the warmer, and a second portion 266B that instructs the user to press the start button on the warmer to begin the reaction.

FIG. 2E is an exemplary interface 268 showing results steps, according to one embodiment. The interface 268 includes a first portion 268A instructing a user to grab the dropper by the tip and snap their wrist down to collect the liquid in the bottom of the dropper, a second portion 268B that instructs the user to remove the warmer tube from the warmer and place it into the chimney of its corresponding reader, a third portion 268C that instructs the user to squirt the entire contents of the dropper into the center of the reader chimney, and a fourth portion 268D that instructs the user to insert the tube into the reader and to press down using both thumbs on the tube. The fourth portion 268D further instructs the user that the user may hear a pop sound, to press until the top is flush with the lip of the reader chimney, and to tap the reader against the work surface three times and to start a timer for five (5) minutes to allow the reader to develop.

FIG. 2F shows an exemplary interface 270 for reading results of the test, according to one embodiment. The interface 270 includes three exemplary reads, including read 270A for a positive test (in this example, for SARS-CoV-2), read 270B for a negative test, and read 270C for a test that was not performed correctly, and therefore is invalid.

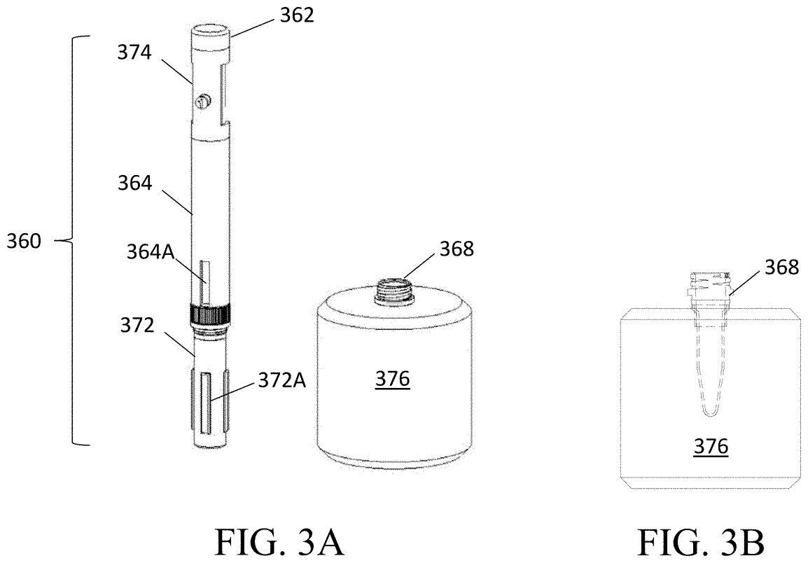

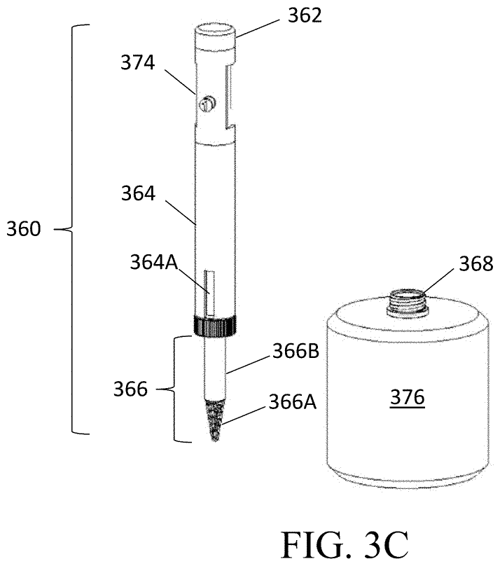

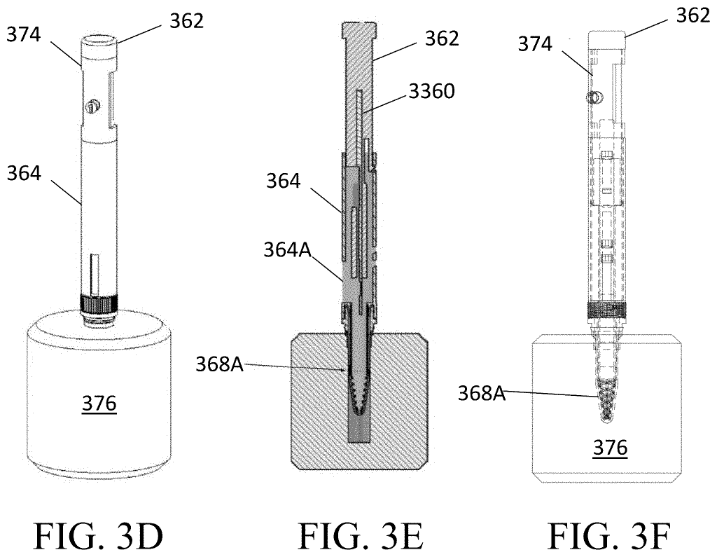

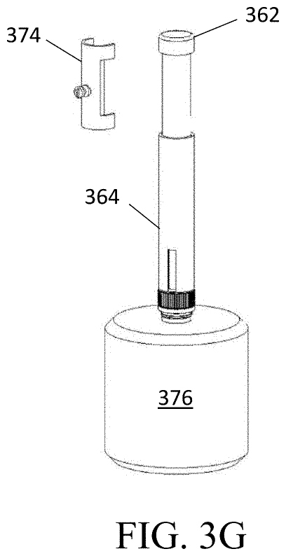

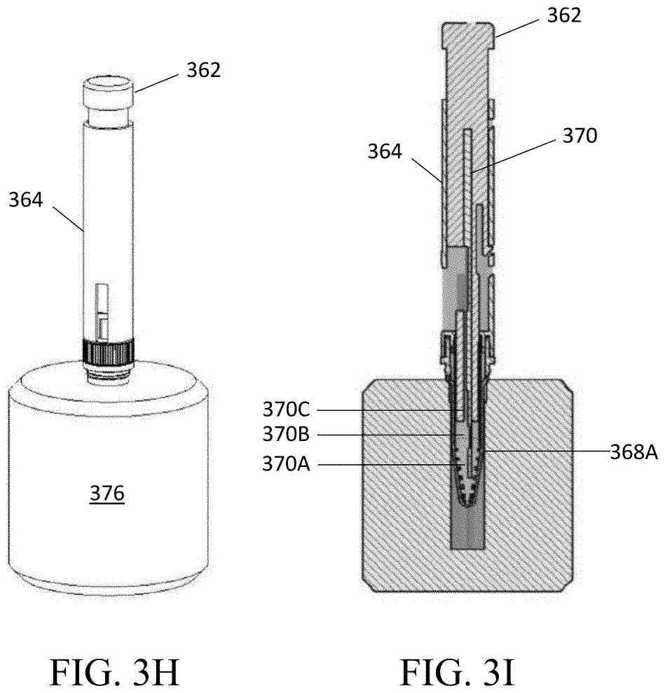

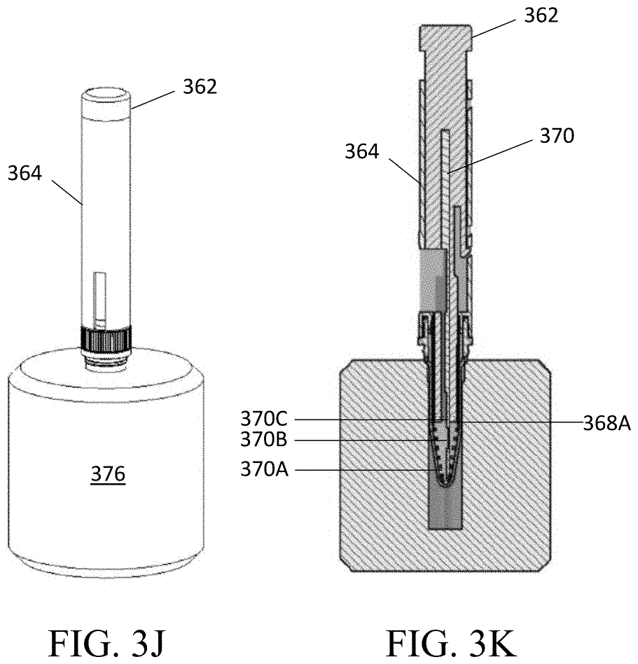

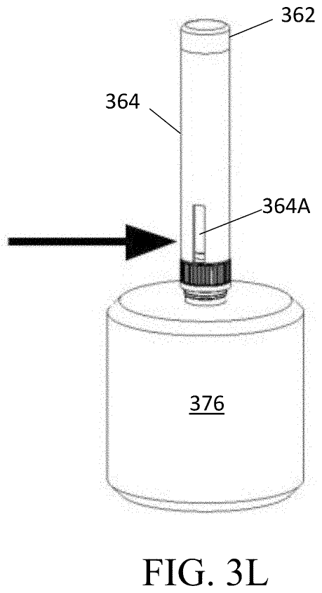

Another exemplary embodiment of a diagnostic testing method is shown in FIGS. 3A-3L. The downloadable software application can be configured to provide instructions to perform the diagnostic testing method shown in FIGS. 3A-3L. As shown in FIG. 3A, a user may remove a cap of reaction tube 368 and place reaction tube 368 in heating unit 376. One or more protruding elements 372A on removable cap 372 of diagnostic device 360 may prevent removable cap 372 from being mistakenly inserted into reaction tube 368 and/or heating unit 376. FIG. 3B shows a cross-sectional view of reaction tube 368 in heating unit 376.

As shown in FIG. 3C, a user may remove cap 372 from diagnostic device 360, exposing sample-collecting component 366, which comprises swab element 366A and stem element 366B. In some embodiments, cap 372 may be configured to hold reaction tube 368 (e.g., during sample collection, prior to placing reaction tube 368 in heating unit 376). Sample-collecting component 366 may then be used to collect a sample (e.g., collecting a nasal secretion, oral secretion, cell scraping, blood, or urine by inserting sample-collecting component 840 into a nasal or oral cavity of a subject).

As shown in FIGS. 3D-3F, after a sample has been collected, diagnostic device 360 may be inserted into reaction tube 368 in heating unit 376 such that at least a portion of swab element 366A is in physical contact with fluidic contents 368A of reaction tube 368. FIG. 3D shows an external view, and FIGS. 3E-3F show cross-sectional views, of diagnostic device 360 inserted into reaction tube 368, which is in heating unit 376. FIGS. 3E and 3F show that at least a portion of swab element 366A of sample-collecting component 366 is in physical contact with fluidic contents 368A of reaction tube 368. In addition, FIGS. 3E and 3F show that substrate 370 is not in physical contact with fluidic contents 368A of reaction tube 368. In certain embodiments, diagnostic device 360 may be screwed into or otherwise fastened to reaction tube 368 and/or heating unit 376 to provide a more secure connection.

As shown in FIG. 3G, safety clip 374 may be removed from diagnostic device 360. The removal of safety clip 374 may allow inner component 362 to move relative to outer component 364.

As shown in FIGS. 3H and 3I, one or more motions may be performed to move inner component 362 relative to outer component 364. The one or more motions may comprise pushing inner component 362 a first distance into outer component 364. FIG. 3H shows an external view of diagnostic device 360 and heating unit 376 after inner component 362 has been pushed a first distance into outer component 364. In some embodiments, the one or more motions result in a first portion of substrate 370 (e.g., reagent delivery region 370A) contacting fluidic contents 368A of reaction tube 368. FIG. 3I shows a cross-sectional view of diagnostic device 360 after inner component 362 has been moved relative to outer component 364 such that reagent delivery region 370A is in contact with fluidic contents 368A of reaction tube 368. As a result, fluidic contents 368A of reaction tube 368 may comprise one or more reagents (e.g., lysis reagents, reverse transcription reagents, nucleic acid amplification reagents, CRISPR/Cas detection reagents) dissolved in a reaction buffer. As further shown in FIG. 3I, substrate 370 further comprises separation region 370B and lateral flow assay region 370C. Lateral flow assay region 370C is not in physical contact with fluidic contents 368A of reaction tube 368, and separation region 370B prevents any liquids from being transported to lateral flow assay region 370C.

In some embodiments, heating unit 376 may heat fluidic contents 368A of reaction tube 368 to one or more desired temperatures for a desired amount of time. In certain instances, heating fluidic contents 368A of reaction tube 368 may facilitate lysis of cells in the sample (e.g., via thermal or chemical lysis). In certain instances, heating fluidic contents 368A may facilitate reverse transcription of RNA in the sample (e.g., viral RNA) to DNA (e.g., cDNA). In certain instances, heating fluidic contents 368A may facilitate amplification of nucleic acids (e.g., via LAMP, RPA, tHDA, NASBA, or NEAR). As a result, after heating, fluidic contents 368A may comprise amplified nucleic acids (i.e., amplicons).

As shown in FIGS. 3J and 3K, one or more additional motions may be performed to further move inner component 362 relative to outer component 364. The one or more additional motions may comprise further pushing inner component 362 a second distance into outer component 364. FIG. 3J shows an external view of diagnostic device 360 and heating unit 376 after inner component 362 has been pushed a second distance into outer component 364. In some embodiments, the one or more additional motions result in a second portion of substrate 370 (e.g., lateral flow assay region 370C) contacting fluidic contents 368A of reaction tube 368. FIG. 3K shows a cross-sectional view of diagnostic device 360 after inner component 362 has been moved relative to outer component 364 such that at least a portion of lateral flow assay region 370C is in contact with fluidic contents 368A of reaction tube 368. From FIG. 3K, it can be seen that reagent delivery region 370A, separation region 370B, and lateral flow assay region 370C are all in physical contact with fluidic contents 368A of reaction tube 368 after the one or more additional motions have been performed. In some cases, contact between lateral flow assay region 370C and fluidic contents 368A may allow amplicons in fluidic contents 368A to travel via capillary action through lateral flow assay region 370C, which may comprise one or more test lines comprising one or more capture reagents (e.g., immobilized antibodies) configured to detect one or more target nucleic acids. In some cases, lateral flow assay region 370C may further comprise one or more control lines comprising a human nucleic acid control and/or a lateral flow control. In some cases, if the one or more target nucleic acids are present in the sample, the one or more test lines will become detectable. In some cases, if a sample has been properly collected and/or the diagnostic test has been properly run, the one or more control lines will become detectable. As shown in FIG. 3L, the one or more lines on substrate 370 (e.g., in lateral flow assay region 370C) may be detectable when opening 364A in outer component 364 is aligned with an opening in inner component 362.

It should be appreciated that the foregoing exemplary diagnostic devices, tests and test steps discussed in conjunction with FIGS. 2A-2F and FIGS. 3A-3L are for illustrative purposes and are not intended to be limiting. Further examples of diagnostic devices, tests, and test kits are described herein. Therefore, the downloadable software application is not limited to such aspects, and can be used with any test, diagnostic device, or test kit.

II. Software-Based Ecosystem

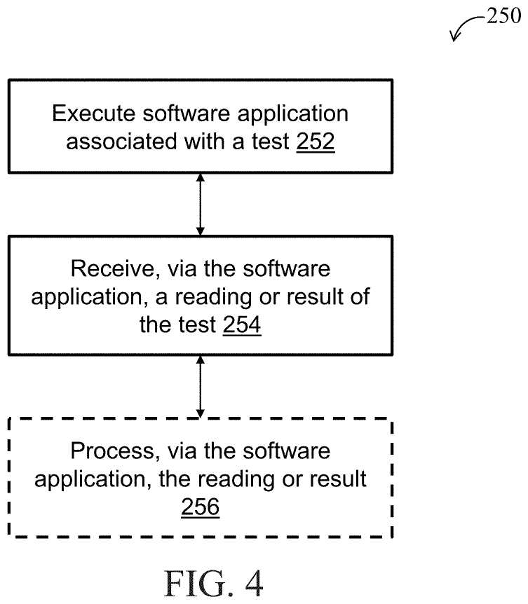

In an embodiment, the techniques include a test ecosystem including a home test kit for testing for a viral illness such as COVID19, influenza type A and/or influenza type B, and an ecosystem configured to integrate test readings, test results and/or other information. In an embodiment, the testing ecosystem stores test information and other information in a central database, and can disseminate information to other devices, including clinician databases/devices, agency databases/devices, medical record databases/devices, and/or the like. In an embodiment, the ecosystem can integrate aspects of patient health relating to disease progression. In an embodiment, the testing ecosystem can incorporate data from other data sources, including data provided by the users and/or data available from other data sources (e.g., clinician databases/devices, agency databases/devices, medical record databases, and/or the like). In an embodiment, the testing ecosystem stores tracking data for users of the testing ecosystem that the testing ecosystem can use to provide additional services (e.g., contact tracing). In some embodiments, a software application can process test results (e.g., read, receive, analyze and/or generate the test results) and upload the results to the software-based ecosystem. Test results may be uploaded, manually or automatically, to the device and/or to a networked device. In an embodiment, the test reading and/or result is uploadable to a device running the software application which can upload the reading or result to the ecosystem. FIG. 4 is an exemplary computerized method 250 for receiving and/or processing a test reading and/or result, according to some embodiments. Aspects of FIG. 4 can be performed by the software application and/or the software-based ecosystem. At step 252, the device executes a software application associated with the test. At step 254, the device receives, via the software application, a reading or result of the test. In an embodiment, the test reading is of a rapid test for COVID 19. In an embodiment, the test reading is of a rapid test for influenza. In an embodiment, the test reading is of a rapid test for influenza A or influenza B. In an embodiment, the test reading is of a rapid test for a target nucleic acid. In an embodiment, the software application receives and processes results of an antibody or antigen test. At step 256 the software application can optionally analyze a test reading and/or test results. In an embodiment, the downloadable software application provides a test result automatically from the entered test reading.

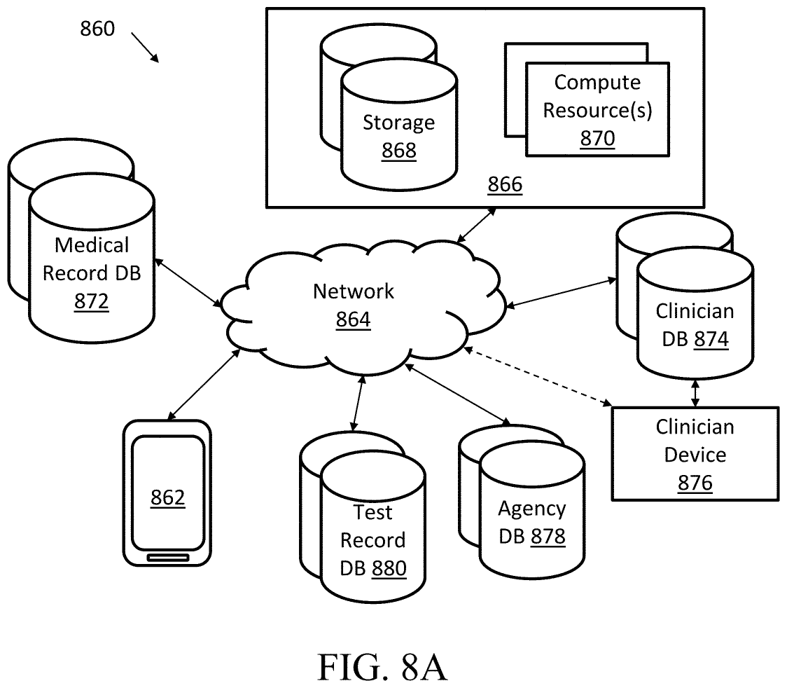

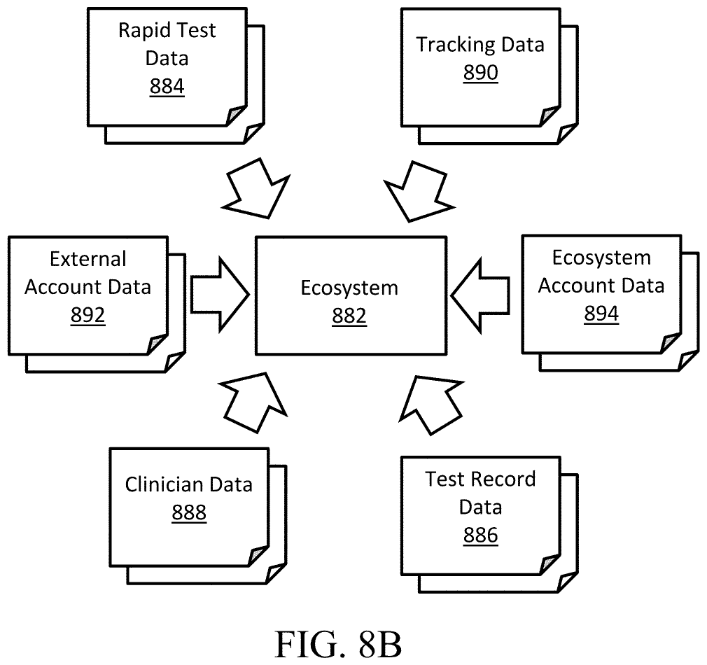

FIG. 8A is a diagram of exemplary components of a testing ecosystem 860, according to some embodiments. The testing ecosystem 860 includes a device 862 that downloads a software application that, when executed by the device 862, is configured to guide a user through administration of the testing. The software application is further configured to upload test results and/or other subject data (e.g., subject age, subject health information, subject location data, other testing data, and/or the like), manually or automatically, to one or more of the components of the testing ecosystem 860. The device 862 can be in communication with the component(s) through the network 864 and/or may be in direct wired and/or wireless communication with the component(s).

The components of the testing ecosystem 860 include one or more resources 866, which can include storage 868 and/or compute resource(s) 870. According to some embodiments, the resource 866 is used to aggregate subject data, including testing data as well as other data (e.g., from the rapid test and/or other test(s)). According to some embodiments, the resource 866 is a remote server, a back-end server, a cloud resource, and/or the like. In some embodiments, the storage 868 of the resource 866 can provide a central database for the testing ecosystem that can be used to store user information, account information, medical information, and/or the like.

The components of the testing ecosystem 860 also include other computing resources, including one or more medical record databases 872, one or more clinician databases 874, one or more agency databases 878 (e.g., the Center for Disease Control, state and/or federal authorities), and one or more test record databases 880 (e.g., HIPAA-compliant databases), in this example. In an embodiment, users of the database(s) can access the databases via user devices. For example, as shown in FIG. 8A, a clinician can access the clinician database 874 through a clinician device 876, which can include a smartphone, tablet, laptop, and/or any other type of computing device. In some embodiments, the testing ecosystem can allow a device 862 to communicate test results and/or other data directly to user devices, such as directly to a clinician device 876 (e.g., without needing to store the data in the clinician database 874).

In some embodiments, at least a portion of the testing ecosystem 860 can be implemented using a cloud-based infrastructure. For example, at least a portion of the resources 866 (including the storage 868 and/or compute resources 870) can be implemented partially and/or entirely using a cloud-based infrastructure. As another example, some or all of the resources 866, medical record database 872, clinician database 876, agency database 878 and/or test record database 880 can be implemented using one or more cloud-based infrastructures. In some embodiments, aspects of the testing ecosystem 860 can be implemented as a secure, HIPAA-compliant, cloud-based software infrastructure. As such, users (e.g., via the mobile application) can send information (e.g., user information, test information, such as an image of the resultant lateral flow test strip, etc.) to a HIPAA-compliant architecture. The software infrastructure can facilitate simple, fast, and scalable reporting and/or other communication with users (e.g., users via devices 862), clinicians (e.g., via clinician devices 876), hospitals or other medical providers (e.g., via medial record database 872), healthcare networks, insurance companies, federal health agencies, state health agencies, and/or the like.

Data can be communicated among the components of the testing ecosystem 860 in any format. In some embodiments, components of the testing ecosystem 860 may store data in different formats. For example, storage components of the testing ecosystem 860, such as storage 868, medical record database 872, clinician database 876, agency database 878 and/or test record database 880, can use various (and different) databases and data storage structures. For example, the data can be stored as structured or unstructured data in one or more databases (e.g., using SQL, NoSQL, MongoDB, Hadoop, Oracle, and/or the like). As another example, the data can be stored as electronic health records (EHRs), electronic medical records (EMRs), and/or using one or more EHR and/or EMR software platforms (including custom platforms and/or platforms provided by third party vendors). The components of the testing ecosystem can also be configured to communicate using different wired and/or wireless protocols. For example, components can communicate over telephone networks, cellular networks, cable networks, fiber-optic networks, the Internet, and/or the like. As a result, it should be appreciated that various communication protocols can be used among components of the testing ecosystem 860. Exemplary communication protocols include Hypertext Transfer Protocol (HTTP), Ethernet, Internet Protocol (IP), File Transfer Protocol (FTP), Transmission Control Protocol (TCP), User Datagram Protocol (UDP), Simple Mail Transfer Protocol (SMTP), Bluetooth (e.g., Bluetooth Low Energy (BLE), Bluetooth Mesh Networking, etc.), Cellular (e.g., 2G, 3G, 4G, 5G), WiFi (e.g., IEEE 802.11 protocols), ZigBee, Near Field Communication (NFC), Radio Frequency Identification (RFID), and/or the like.

It should be appreciated that while FIG. 8A shows exemplary components that can be part of the testing ecosystem, such illustrative components are not intended to limit the techniques described herein. Similarly, while FIG. 8A shows exemplary communication paths, such illustrated communication paths are not intended to be limiting. The ecosystem can provide various communication path(s) to various resources(s) for the user to upload data to various places within the ecosystem. In some embodiments, the data can be first uploaded to a first storage location (e.g., a central database of the ecosystem) and then routed, by the ecosystem (e.g., via software running on a server device of the ecosystem) to one or more additional devices. Therefore, the software application may provide data to one or more devices in the ecosystem, and that data can be automatically transmitted to other devices in the ecosystem.

FIG. 8B is a diagram showing examples of various data that can be integrated into a testing ecosystem 882, according to some embodiments. As described herein, the ecosystem 882 can be configured to generally integrate all aspects of patient health relating to disease progression, including rapid test results, health information, antibody testing, and/or other relevant patient information. As shown in FIG. 8B, the ecosystem 882 is configured to integrate rapid test data 884. The rapid test data 884 can include data associated with one or more rapid tests, including test readings and/or test results from the one or more rapid tests. For example, a user of the ecosystem may test themselves one or more times, and the ecosystem can integrate all of the rapid test data into the ecosystem.

The ecosystem 882 can receive test record data 886. The test record data can include data for one or more other tests. The other tests can include, for example, tests performed by a third party, such as tests performed at a testing site, tests performed by a clinician, etc. In some embodiments, the test record data can include antibody test data, COVID-19 test data, influenza test data, and/or target nucleic acid test data. The ecosystem 882 can receive clinician data 888. The clinician data 888 can include patient health data, historical patient data, medical examination data, surgical data, patient medical records, and/or any other clinical patient data. The ecosystem 3560 can receive tracking data 890. In an embodiment, the tracking data can include user location data, residence data, address data, GPS-based data, and/or other tracking data available for a user.

The various data integrated into the test eco system 882 can be accessed various ways. In some embodiments, data can be entered manually by a user. For example, the user can enter data via an interface, such as a web interface or software app configured to communicate with the ecosystem 882. In some embodiments, data can be entered by a user through uploading an image of the data (e.g., images of test readings, medical records, etc.). In some embodiments, the ecosystem 882 can determine one or more portions of the data of the ecosystem. For example, the ecosystem 882 can receive a test reading and determine the test result.

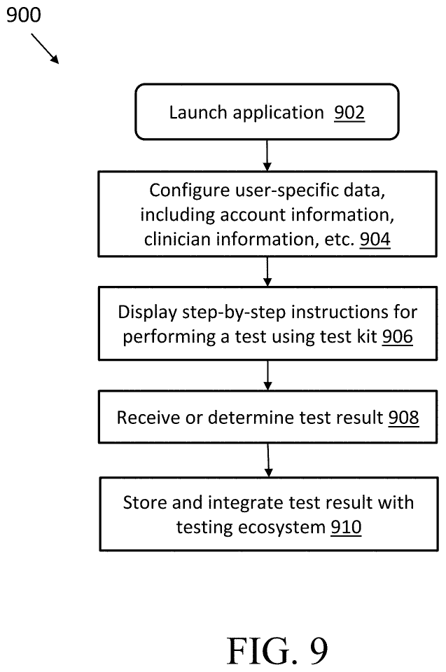

In some embodiments, the ecosystem 882 can be configured to automatically interact with one or more external data resources, such as a testing resource, a clinician computing resource, a medical record computing resource, and/or an agency computing resource (e.g., as discussed in conjunction with FIG. 8A). The ecosystem 882 can receive external account data 892 for accessing one or more external data resources. For example, a user may input a URL and associated access information as part of the external account data 892 so that the ecosystem 882 can pull data from the data source accessible through the external account data 892. In some embodiments, the user can store ecosystem account data 894. As described herein (e.g., in FIG. 9), for example, rapid test kit users can create accounts with the ecosystem.

Users of the ecosystem can create accounts with the ecosystem and store information within the ecosystem. In some embodiments, the software application is configured to guide a subject through setting up an account with the ecosystem (e.g., where the subject may be the user, for a self-administered test) and performing a test. FIG. 9 is a diagram of an exemplary computerized method 900 for configuring user-specific data and integrating a test result into the testing ecosystem, according to some embodiments. At step 902, the user launches the software application (e.g., via device 862). At step 904, the software application allows a user to setup and/or manage user-specific data for the testing ecosystem. For example, the user can configure ecosystem account data 894 as discussed in conjunction with FIG. 8B. In some embodiments, the user can create and/or sign into an account with the testing ecosystem. The user can manage information associated with the user's account, including personal data, healthcare data, and/or other data. The personal data can include the user's name, social security number, date of birth, address, phone number, email address, medical history, medications, and/or the like. The healthcare data can specify one or more clinicians of the user (e.g., physician(s), psychiatrist(s), psychologist(s), nurse(s), and/or other doctors or medical professionals that care for the user). The healthcare data can include data provided by the user of the software application. In some embodiments, the software application can provide forms for users to enter healthcare information, can allow a user to take an image of healthcare information, and/or the like, to provide the information to the ecosystem.

At step 906, the software application provides, via the device 862, instructions for using a diagnostic device and/or otherwise performing a diagnostic test method. In an embodiment, the application will guide a user through detailed steps, described herein, for implementing the test.

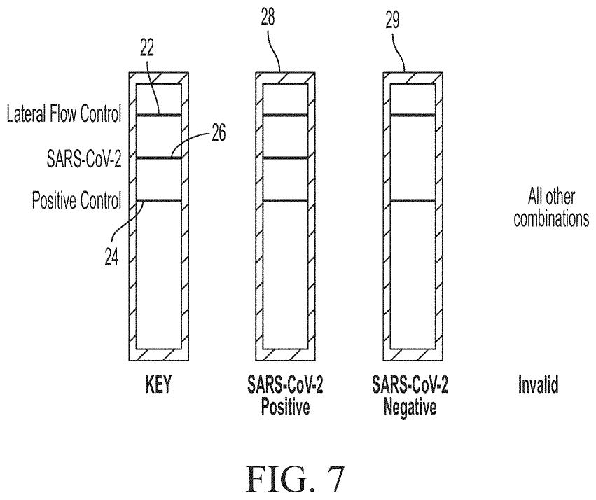

At step 908, the software application receives and/or determines a test result for an administered diagnostic test. The software application can be used to read and analyze test results. Such an embodiment of the software application for reporting and analyzing results is illustrated in FIG. 2F and FIGS. 6A-7. As shown in FIG. 7, in an embodiment, the readout device includes a lateral flow control line 402, a positive control line 404, and a test target (e.g., SARS-CoV-2, influenza, other pathogen) line 406. The lateral flow control line 402 and positive control line 404 are to illustrate whether the test was performed and accomplished accurately. Only if both control lines 402, 404 are positive and dark (highlighted) was the test performed and accomplished accurately. If either control line is not positive and dark, then the test is invalid and must be repeated. Only if all three lines are positive and dark is the test positive (for COVID-19, influenza, or some other target nucleic acid), as illustrated by positive reading 408. If the test target line 406 is not positive and dark, then the test is negative as illustrated by negative test reading 410.

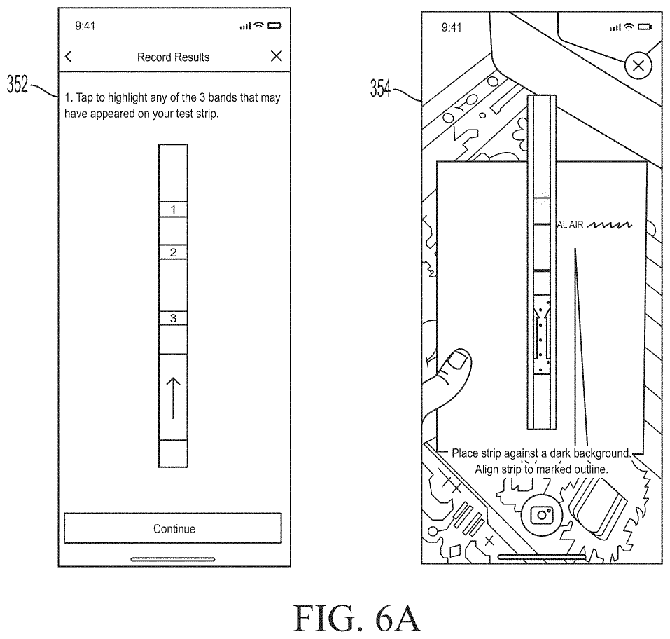

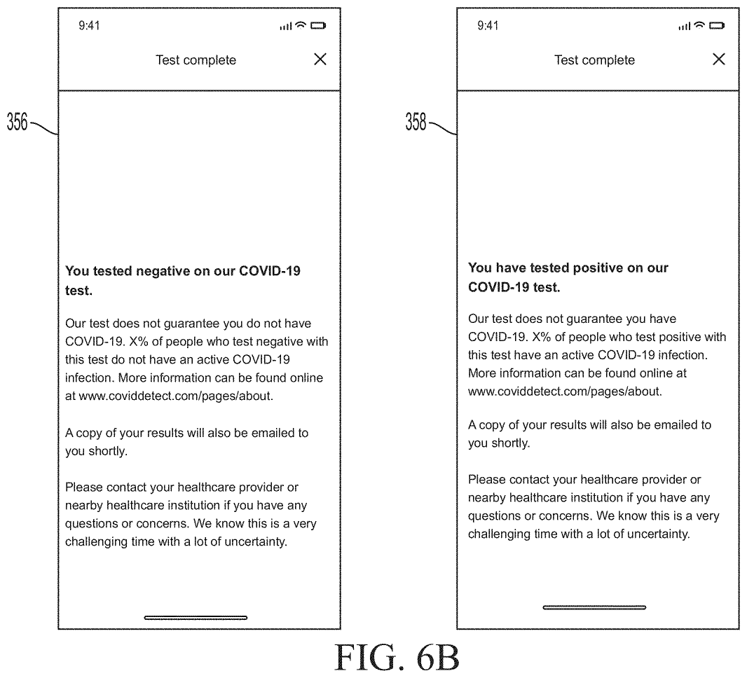

In an embodiment, the downloadable software application provides a user with the ability to enter a test reading or result. The test can be self-read, read by another, or uploaded to a device containing the software application for automatic reading. In an embodiment, a user manually enters the reading or result to the downloadable software application. For example, through an image or user interface appearing on a device containing the software application, a user can tap the number of lines (bands) appearing positive on the readout strip and the software application will automatically read the results. This is shown in the interface 352 in FIG. 6A. Alternatively, a user may take an image of the readout strip and upload that image to the device containing the software application for automatic reading of the test results. This image taking application is illustrated in the interface 354 in FIG. 6A. In the interface 354 in FIG. 6A, a marked outline is displayed on the portable electronic device to help the user align the image to the detection component of the diagnostic test prior to capture. In some embodiments, the user may capture the photo by selecting a button (e.g., the camera icon shown in interface 354). In some embodiments, the image may be captured automatically (e.g., when the detection component is aligned with a corresponding outline, or when the detection component is automatically detected in the image). In an embodiment, the test reading and/or result is uploaded through a wireless connection.

In some embodiments, as described herein, a device (e.g., a camera, a smartphone) is used to generate an image of a test result (e.g., one or more lines detectable through openings in the inner and outer components). According to some embodiments, the techniques can automatically determine the results of a diagnostic test based on an image of a detection component of the diagnostic test. As described herein, a diagnostic test may include steps of collecting a sample from a subject (e.g., a human being, such as a patient being tested for a disease), processing the sample (e.g., with any of the processing techniques described herein), and analyzing the sample with the detection component. For example, a colorimetric assay or test strip, such as a lateral flow test strip, may be used to analyze the sample and indicate, via lines on the test strip, colors on the colorimetric assay, or any other suitable indicator, visual information regarding the results of the test. A computer vision software application may be employed to read the uploaded or entered test reading, and automatically provide a positive or negative test result as described further herein.

The results of a test, once determined according to method 450 or by any other means, may be communicated directly to the user or directed to another, such as a medical professional. The test results can be communicated to a central database server of the software-based ecosystem and/or to a remote doctor or other. Referring further to FIG. 9, at step 910 the software application stores and integrates the test result with the testing ecosystem. For example, referring to FIGS. 8A-8B, according to some embodiments components of the testing ecosystem 860 (e.g., the storage 868 of the resources 866, medical record database 872 clinician database 876, and/or agency database 878) can store test results, user information, and/or patient information (e.g., rapid test data 884, test record data 886, and clinician data 888 in FIG. 8B). In some embodiments, the storage 868 is part of a server that is configured to provide a central database to store patient information for test subjects. In some embodiments, the patient information includes an electronic medical record. As described herein, the patient information can include the patient's name, social security number, date of birth, address, phone number, email address, medical history, medications, etc. In some embodiments, the patient information includes information from one or more remote data sources, such as clinician database 874 and/or medical record database 872.

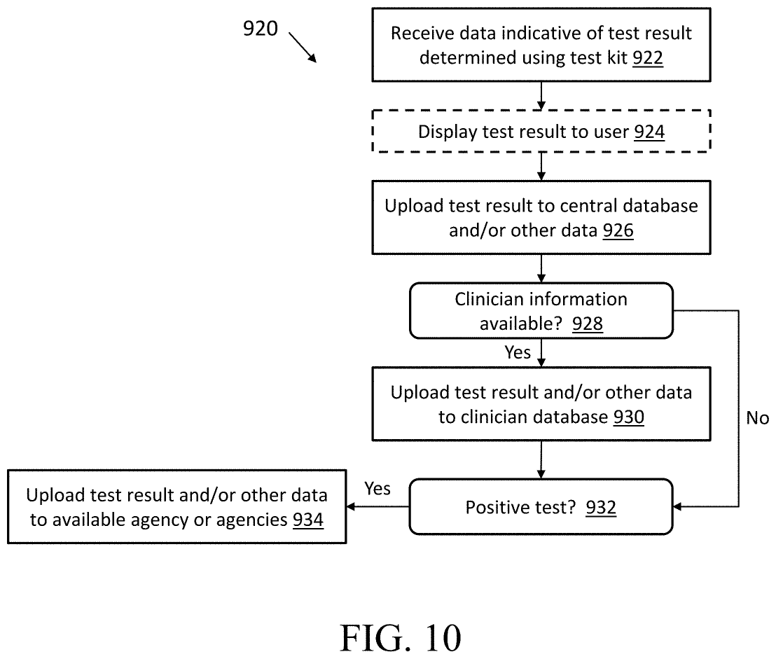

As shown in FIG. 8A, the software application can upload (via the user device) data to one or more devices that are part of the ecosystem. FIG. 10 is a flow chart of an exemplary computerized method 920 for uploading test result data and/or other data to the ecosystem, according to some embodiments. At step 922, the software application (e.g., running on the user's device) receives data indicative of a test result determined using the diagnostic test kit. At step 924, the software application can optionally display the test result to the user. For example, if the software application determines the test result (e.g., using machine vision), the software application can display the determined test result to the user. At step 926, the software application uploads the test result to a central database and/or other devices of the ecosystem.

At step 928, the software application determines whether clinician information is available for the user. For example, the software application can determine whether the central database stores information for the clinician, such as a clinician name, a practice name, telephone number, fax number, database and/or server information, log-in information, and/or the like, for the clinician (e.g., as part of external account data 892 discussed in FIG. 8B). If clinician information is available, at step 930 the software application uploads/sends the test result and/or other data to the clinician(s) and/or clinician databases. For example, the software application can send a notification to the clinician via text message. As another example, the software application can upload the information to a clinician database so that the clinician can store the information in the clinician's files for the user and/or access the information through applications used by the clinician.

At step 932, the software application determines whether the test is a positive test. If the test result is positive, at step 934 the software application can upload the test results to the test result and/or other data to an applicable agency, such as the Board of Health, a monitoring center, and/or the like. The ecosystem may be pre-configured with the applicable agency data and/or can be configured to determine the agency data based on information about the user (e.g., based on the user's location, etc.). Therefore, the user may not be required to provide the agency data in order for the ecosystem to provide information indicative of a positive test result to the applicable agencies.

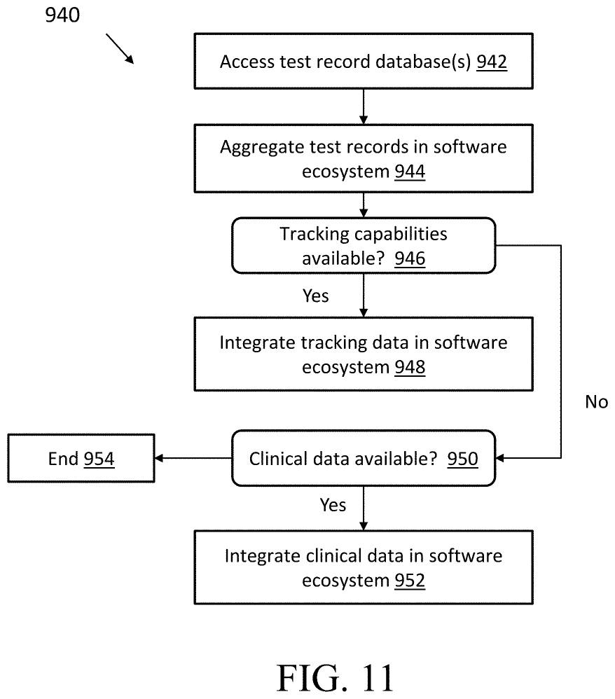

In some embodiments, the ecosystem can aggregate and store user information. FIG. 11 is a flow chart of an exemplary computerized method 940 for aggregating user information, according to some embodiments. At step 942, the ecosystem (e.g., via compute resources 870) accesses one or more test record databases and/or other test data sources for the user. At step 944, the ecosystem aggregates the accessed test records into the software ecosystem. For example, the user can link test records performed by various testing facilities, tests for different viruses and/or target nucleic acids. As another example, the user can link to test for antibodies. As a result, the ecosystem can provide an integrated set of tests, test results and other data for a particular user so that the user can view and access all test results and other data relevant to disease progression through the ecosystem.

At step 946, the ecosystem determines whether user tracking capabilities are available for the user. In some embodiments, the system can include user or patient tracking capabilities, such as with use of smartphones or remote devices with tracking capabilities, IP address monitoring, and/or the like. At step 948, the ecosystem (e.g., via the use of the software application running on user device(s)) integrates tracking data into the ecosystem (e.g., tracking data 890 discussed in conjunction with FIG. 8B). In some embodiments, the tracking information may also be communicated to a central database server and/or to a remote doctor or other. Thus, user tracking information (e.g., location information over time) can be tracked and monitored by the ecosystem.

In some embodiments, the ecosystem can assess and notify others who come into contact or within a certain distance of any user, such as a user who has tested positive for a viral illness.

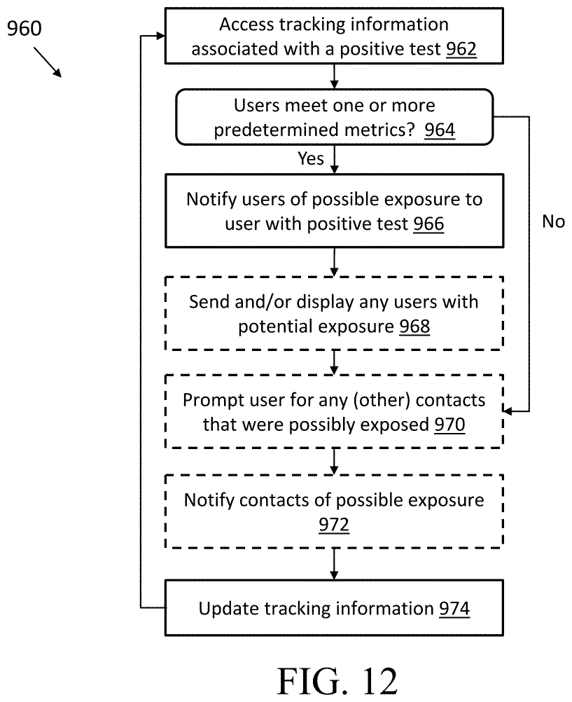

FIG. 12 is a flow chart of an exemplary computerized method 960 for performing contact tracing, according to some embodiments. At step 962, the ecosystem (e.g., compute resources 870) accesses tracking information associated with a positive test (e.g., via storage 868). The tracking information can include, for example, location information for a user with a positive test. At step 964, the ecosystem can search a tracking database (e.g., stored via storage 868) to determine whether one or more other users meet any predetermined metrics based on the tracking information for the positive test. For example, the ecosystem can identify one or more other users with tracking information that is indicative of the user being within a certain proximity of the positive user. As another example, the ecosystem can identify one or more other users with tracking information that is indicative of the user being within a certain proximity of the positive user at one or more predetermined time periods (e.g., within a certain number of days prior to the user testing positive, within a certain number of days after the user testing positive, and/or the like).

At step 966, if the ecosystem identified one or more users that meet the one or more predetermined metrics, the ecosystem can notify the users of possible exposure to the user with the positive test. The ecosystem can notify the users via text message, phone, fax, messaging through the software interface, and/or the like. At step 968, the ecosystem can optionally send and/or display information regarding any users with potential exposure. For example, the ecosystem can send and/or display the information to a user with a positive test result, to medical professional(s), to an agency or agencies, and/or the like. In some cases, a user's test results, information, and/or location may be communicated to state and/or federal health agencies. The information can include names, contact information, tracking data, and/or the like. In some embodiments, the information is general information that cannot be used to identify individual people, depending on confidentiality requirements and/or user preferences.

The method 960 next proceeds to step 970 (from either step 966/968 or from step 964 if the ecosystem does not identify any users that meet the one or more predetermined metrics). At step 970, the ecosystem can optionally prompt a user with a positive test result for information on any contacts that the user believes may have been potentially exposed to them. For example, the user can enter in any contacts not identified at step 968 that the user believes may have met one or more of the predetermined metrics (e.g., proximity and/or time). As another example, the user can simply enter all people that the user believes may have met one or more of the predetermined metrics, and the ecosystem can de-duplicate that information with any users identified at step 964. At step 972, the ecosystem can notify contacts of possible exposure. For example, the ecosystem can use provided contact information (e.g., phone number, email address, etc.) to automatically notify the user of potential exposure. As another example, the ecosystem can provide the data entered by the user at step 970 to appropriate agencies or authorities, who can in-turn contact the people using the provided information and/or other available contact information that is identifiable for those individuals. At step 974, the ecosystem can update the tracking information accessed at step 962, and proceed back to step 962 to repeat the method 960.

Referring further to FIG. 11, at step 950, the ecosystem determines whether clinical data is available for the user (e.g., clinical data 888 discussed in conjunction with FIG. 8B). In some embodiments, the clinical data can be accessed based on information provided by the user (e.g., clinician information, log-in information for databases and/or platforms used by the clinician, etc.). In some embodiments, the user may provide clinical data to the ecosystem (e.g., by uploading the data to the ecosystem, by capturing images of printouts of clinical data, etc.). At step 952, the ecosystem integrates the clinical data in the ecosystem. For example, the ecosystem can integrate the test results into a central database of the ecosystem (e.g., provided by storage 868 as shown in FIG. 8A). Otherwise, if clinical data is not available, the method can end at step 954.

In some embodiments, the database may generate a code based on the user's results (e.g., positive or negative for the viral illness). After a successful test, the code is available in the application. In some embodiments, the code is read by a bar code scanner or other security detection device. If the user is negative or the viral illness and has a negative code, the security system will recognize the code and permit entry. In other embodiments, if the user is positive for the viral illness and has a positive code, the security system will recognize the code and deny entry.

In some embodiments, the database may generate a code based on the user's results (e.g., positive or negative for the viral illness). After a successful test, the code is available in the application. In some embodiments, the code is read by a bar code scanner or other security detection device. If the user is negative or the viral illness and has a negative code, the security system will recognize the code and permit entry. In other embodiments, if the user is positive for the viral illness and has a positive code, the security system will recognize the code and deny entry.

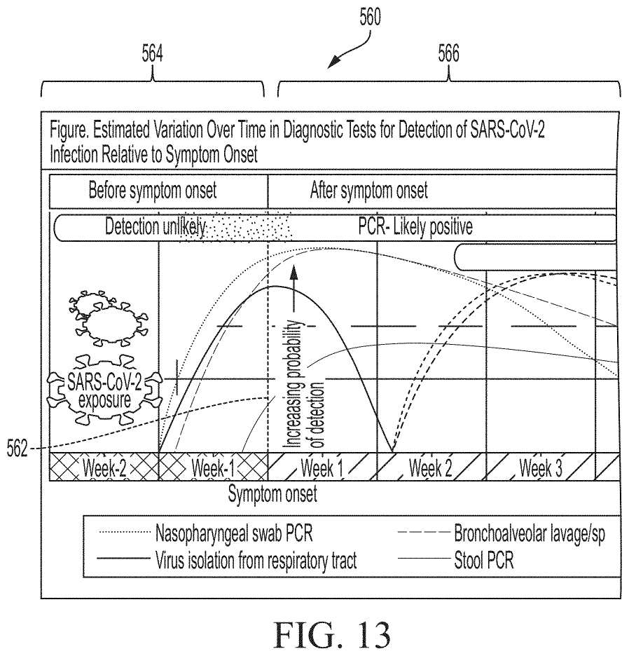

FIG. 13 illustrates a timeline 560 associated with diagnostic and antibody testing of a patient with SARS-CoV-2 infection. The vertical line 562 represents symptom onset, such that the two weeks in section 564 occur prior to symptom onset, while the three weeks in section 566 are after symptom onset. Shown are the likelihood of positive detection tests for several different types of test detection approaches versus time, including prior to and after symptom onset. Also shown is the timeline of antibody detection of various types.

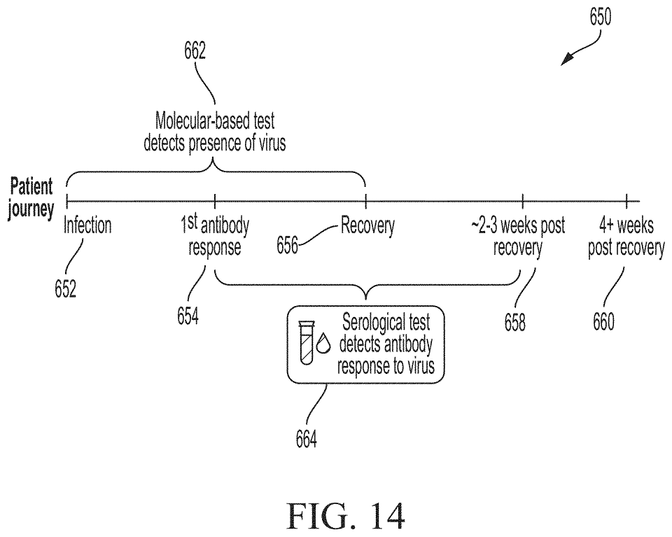

FIG. 14 is a timeline 650 of patient infection showing integration into a software-based application for receiving and processing disease test results and serological antibody test results, as described. The timeline 650 includes patient infection 652, patient antibody response 654, patient recovery 656, a period approximately 2-3 weeks post recovery 658, and four plus weeks post recovery 660. As shown at 662, molecular-based testing can be used to detect the presence of the virus between periods 652 through 656, as an example, although other times are not excluded. Similarly, as shown by 664, serological testing can be used to detect antibody response to the virus between periods 654 through 658, although again other times are not excluded. In some embodiments, patient results from a virus test can be digitally integrated with serological tests conducted by a different party via compatible software apps. In some embodiments, the virus test and serological tests can both be provided by a same provider (e.g., offered as a bundle).

It should be appreciated that while some examples of the test kit ecosystem provided herein are discussed in the context of a rapid test and/or other tests described herein, the techniques are not so limited and can be used with any test. Therefore, the examples provided herein of the various tests are intended for exemplary purposes only.

III. Computer Vision

Computer vision techniques can be used to process image data representing detection components of diagnostic tests to obtain corresponding test results. As described herein, these techniques may include a method comprising accessing image data (e.g., stored as pixel values or in any other suitable format) representing a detection component of a diagnostic test (e.g., a lateral flow control test strip, colorimetric assay, or other readout device) and determining, based at least in part on the image data representing the detection component of the diagnostic test, results of the diagnostic test (e.g., a diagnosis of the patient, such as a positive or negative test result for one or more diseases of interest; a validity of the test, such as a valid or invalid test result). In some embodiments, determining the results of the diagnostic test may comprise processing the image data representing the detection component of the diagnostic test with a computer vision algorithm (e.g., a line detection algorithm, an edge detection algorithm, a convolution based algorithm, a machine learning algorithm, or any other suitable algorithm) to obtain an output, and determining the results of the diagnostic test based on the output of the computer vision algorithm.

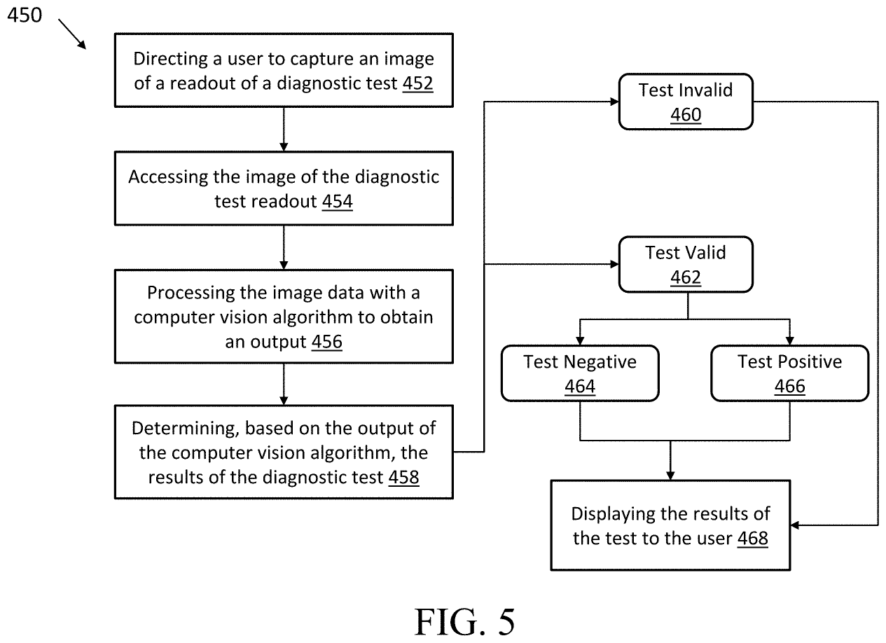

In some embodiments, a diagnostic test comprises or is associated with software to read and/or analyze test results. Such an embodiment of the software application for reporting and analyzing results is illustrated in FIGS. 5-7. As described herein, in an embodiment, the detection component includes a lateral flow control line, a positive control line, and a test target (e.g., SARS-CoV-2, influenza, other pathogen) line, where the lateral flow control line and positive control line are to illustrate whether the test was performed and accomplished accurately. For example, only if both control lines are positive and dark (highlighted) was the test performed and accomplished accurately. In this example, if either control line is not positive and dark, then the test is invalid and must be repeated. In this example, if all three lines are positive and dark is the test positive (e.g., for COVID-19, influenza, or some other target nucleic acid).

The results test can be self-read (e.g., by a clinician and/or by a user), read by another (e.g., by clinicians receiving the results), or uploaded to a device containing the software application for automatic reading, as described herein at least with respect to FIG. 5 and FIGS. 6A-6B. In the example of FIGS. 6A-6B, through an image appearing on a device containing the software application, a user can tap the number of lines (bands) appearing positive on the detection component (e.g., the readout strip) and the software application will automatically read the results. This is shown in interface 352 in FIG. 6A. Alternatively, a user may take an image of the detection component (e.g., the readout strip) and upload that image to the device containing the software application for automatic reading of the test results. This image taking application is illustrated in interface 354 in FIG. 6A.

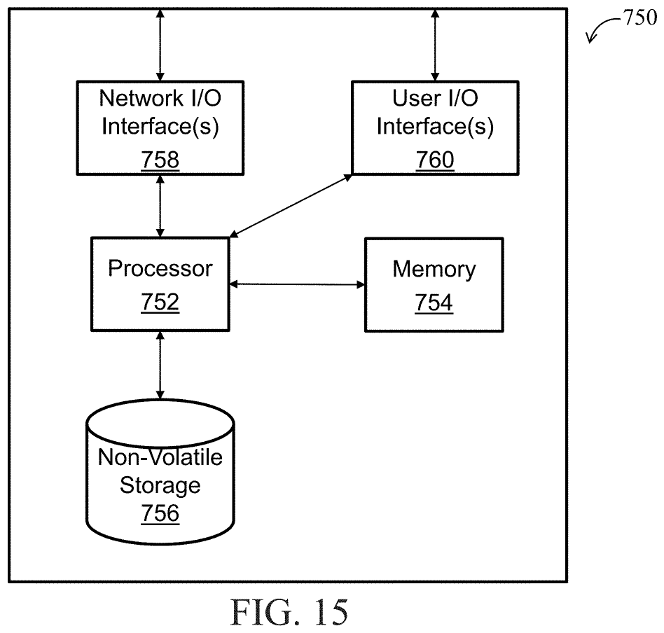

In some embodiments, a device (e.g., a camera, a smartphone) is used to generate an image of a test result (e.g., one or more lines detectable on a lateral flow assay strip). In certain cases, a machine vision software application is employed to evaluate the image and provide a positive or negative test result. FIG. 5 illustrates an exemplary method 450 for determining diagnostic test results, according to some embodiments. The acts of method 450 may be performed with respect to any of the diagnostic tests and/or test kits described herein, or any other suitable diagnostic tests and/or test kits. The acts of method 450 may be a carried out by one or more computer hardware processors, as described herein with respect to FIG. 15.

Method 450 may begin at act 452 with directing a user (e.g., a medical professional administering the test, a patient self-administering the test, or any other individual) to capture an image of a detection component of a diagnostic test (e.g., using interface 354 of FIG. 6A). For example, the user may be directed to use a portable electronic device (such as a smartphone, tablet, or any other suitable device) to capture an image of the detection component of the diagnostic test.

Directing the user to capture an image of the detection component may comprise displaying a message, alert, or interface on a display the portable electronic device. For example, as shown in interface 354 of FIG. 6A, an image capturing interface of the portable electronic device may be overlaid with additional information directing the user to capture the image. In some embodiments, act 452 may further comprises storing the image (e.g., in a data store internal to the portable electronic device capturing the image) and/or transmitting the image (e.g., to an external data store or server, via any suitable connection for transmitting, such as a wired or wireless network connection). The image may be stored and/or transmitted automatically (e.g., without any human input), or may be stored and/or transmitted manually (e.g., with a human providing input, such as with a user interface, to indicate location(s) at which the image is to be stored and/or destination(s) to which the image is to be sent).

The method 450 may continue at act 454 with accessing the image of the detection component of the diagnostic test. For example, the image may be stored in a data store associated with the portable electronic device and accessed from that data store at act 454. Alternatively or additionally, the image data may be received or accessed from an external data store or server. In some embodiments, rather than beginning at act 452, the method 450 may begin at act 454 with the image data being accessed (e.g., the image data may be received from an external source or accessed from an internal or external data store, without needing to be captured by a user). In some embodiments, the image data may be accessed directly from the detection component. For example, the diagnostic test itself may be configured to capture (e.g., with suitable hardware or software elements of the diagnostic test) image data representing the detection component. The image data may be stored, accessed, and/or processed locally (e.g., using a storage medium and/or processor associated with the diagnostic test) or may be transmitted (e.g., to an external data store or server, such as described with respect to act 452) for further processing. In some embodiments, only a portion of the image data may be accessed. For example, the image may be automatically cropped (e.g., by selectively removing, masking, or otherwise ignoring portions of the image data) so as to retain only image data regarding an area of interest (e.g., the area of the image including the detection component).

The method 450 may continue at act 456 with processing the image data with a computer vision algorithm to obtain an output that is used to determine the results of the diagnostic test (e.g., valid, invalid, positive, negative, and/or the like). In some embodiments, the computer vision algorithm may include one or more of a line detection algorithm or an edge detection algorithm (e.g., a Hough transform, a Canny edge detector, or any other suitable technique for line or edge detection). In some embodiments, the computer vision algorithm may include performing feature extraction on the image (e.g., by applying an unsupervised learning technique to the image, or using any other suitable techniques for feature extraction). In some embodiments, the computer vision algorithm may include convolution-based techniques (e.g., including convolution filters which may be applied to pixels of the input image). In some embodiments, the computer vision algorithm may include comparing lines and/or other markings that appear in the image with known patterns of lines and/or markings.

In some embodiments, the computer vision algorithm may include a machine learning model. For example, the computer vision algorithm may include a machine learning model comprising a neural network. In some embodiments, the computer vision algorithm may comprise a neural network having multiple layers (e.g., at least two layers, at least five layers, or at least ten layers) including one or more different types of layers (e.g., convolutional layers, feed-forward layers, pooling layers, dropout layers, or reduction layers). According to some embodiments, the neural network may have many parameters (e.g., at least 100,000 parameters, at least 500,00 parameters, or at least 1,000,000 parameters). The neural network model may be a trained neural network model. For example, the neural network model may be trained on training data including training images (e.g., images of diagnostic tests labeled with the results of that diagnostic test) which may comprise hundreds, thousands, tens of thousands, or more images. According to some embodiments, the training data may be augmented with transformations or otherwise pre-processed as part of training the neural network.

Regardless of which computer vision techniques are employed as part of processing the image at act 456, the output of the computer vision algorithm may include information relating to the detection component of the diagnostic test appearing in the image. For example, the output of the computer vision algorithm may identify a location of the detection component in the image (e.g., with a bounding box or mask). The output of the computer vision algorithm may additionally or alternatively identify locations of features of the detection component, such as lines (e.g., a lateral flow control line, a SARS-CoV-2 line, and/or a positive control line) or other indicators (e.g., dark portions, colors, etc.) which may or may not be visible to a human observer. The output of the computer vision algorithm may additional or alternatively include information such as color/intensity information, a confidence score, or any other information regarding the detection component of the diagnostic test. In some embodiments, if the output of the computer vision algorithm differs from a user input (e.g., an input provided via a user interface, as described with respect to act 452) the user may be notified of the difference, and/or prompted (e.g., by the portable electronic device) to confirm that the user input provided was accurate (e.g., by selecting a button or providing other suitable input indicating confirmation). In some embodiments, for example when the output of the computer vision algorithm differs from a user input, the user may be prompted to repeat act 452 and/or provide additional user input (e.g., to highlight or circle a location of the detection component in the image or tap the locations of particular elements of the detection component in the image).

The method 450 may continue at act 458 with determining, based on the output of the computer vision algorithm, the results of the diagnostic test. As shown in the figure, determining the results of the diagnostic test based on the output of the computer vision algorithm may comprise determining whether the results of the test are valid 462 or invalid 460. For example, in the case of a detection component shown in FIG. 7, the absence of a positive control line 404 indicates that the diagnostic test is invalid. In this example, if the computer vision algorithm output indicates that there is no positive control line on the detection component, then the result of the diagnostic test may be considered invalid 460. Otherwise, if the positive control line is indicated in the computer vision algorithm output, then the result of the test may be considered valid 462. In some embodiments, features other than lines (e.g., colors or other visual indicators) may be used to determine the validity or invalidity of the test based on the output of the computer vision algorithm.

In some embodiments the method 450 may further include determining whether the results of the test are positive 466 or negative 464. According to some embodiments, rather than first determining the validity or invalidity of the diagnostic test result, the method may directly check whether the computer vision algorithm output is indicative of a positive or negative result and thereby infer whether result is valid (e.g., the detection component corresponds to a known positive or negative result, such as in the exemplary positive reading 408 and negative reading 410 of FIG. 7). In some cases, the results may include multiple validity, invalidity, positive, and/or negative results (e.g., for diagnostic tests that test for multiple diseases).

Regardless of the results determined at act 458, the method 450 may continue at act 468 with displaying the results of the diagnostic test to the user. As discussed herein, the results of the test, once determined according to method 450 or by any other means, may be communicated directly to the user or directed to another, such as a medical professional. For example, the results of the diagnostic test may be visually displayed to the user via the portable electronic device used to capture the image of the diagnostic test. The interfaces 356 and 358 of FIG. 6B provide an example of diagnostic test results on a display of a portable electronic device. As shown in the interfaces, the test result may include textual information including links (e.g., links to further information), data regarding the accuracy of the test, or any other relevant information. Further examples of computer vision techniques are described in co-owned U.S. patent application titled “MACHINE VISION TECHNIQUES, INCLUDING IMAGE PROCESSING TECHNIQUES, FOR DIAGNOSTIC TESTING,” also filed on Mar. 16, 2021, which is hereby incorporated by reference herein in its entirety. In some embodiments, the user may be shown a corresponding “Test Complete” screen on the portable electronic device, which may tell the user if the test result is positive, negative, or invalid. In addition to providing the test result, careful language may be used to ensure that the user can properly interpret the meaning of the result. Additionally or alternatively, the results of the diagnostic test may be communicated to the user via other means, such as an audible signal (e.g., spoken words, or a chime, tone, beep, or other noise which may be played, for example, from the portable electronic device).

In some embodiments, the results of the diagnostic test may be communicated to the user via electronic mail, text message, telephone call, physical mail, or any other suitable means of communication. In some embodiments, the results of the diagnostic test may be accessed via an application, such as an application of the portable electronic device or a web application provided by the software-based ecosystem. Accessing the results of the diagnostic test may include requiring the user to verify their identity, such as by providing credentials (e.g., a username and password, biometric information, or other suitable identification). Additionally or alternatively, in some embodiments, the results of the diagnostic test may be transmitted (e.g., via a wired or wireless network connection) to a computing device (e.g., a smart phone, one or more processors arranged in a cloud computing configuration, or any other suitable computing device) for processing.

In some embodiments, acts of method 450 may be omitted, repeated, performed in parallel, or otherwise altered in sequence from the example shown in FIG. 5. For example, the acts relating to capturing the image data may be omitted in some embodiments (e.g., when previously captured image data is received from an external source, rather than being captured on the same device performing the other acts of method 450). In some embodiments, act 468 may be omitted (e.g., if the results are to be transmitted to a remote server, viewed by a medical professional, or otherwise processed other than by displaying them to a user).

IV. Computer Implementation

An illustrative implementation of a computer system 750 that may be used in connection with any of the embodiments of the technology described herein (e.g., such as the downloadable software aspects and/or software-based ecosystem) is shown in FIG. 15. The computer system 750 includes one or more processors 752 and one or more articles of manufacture that comprise non-transitory computer-readable storage media (e.g., memory 754 and one or more non-volatile storage media 756). The processor 752 may control writing data to and reading data from the memory 754 and the non-volatile storage device 756 in any suitable manner, as the aspects of the technology described herein are not limited in this respect. To perform any of the functionality described herein, the processor 752 may execute one or more processor-executable instructions stored in one or more non-transitory computer-readable storage media (e.g., the memory 754), which may serve as non-transitory computer-readable storage media storing processor-executable instructions for execution by the processor 752.

Computing device 750 may also include a network input/output (I/O) interface 758 via which the computing device may communicate with other computing devices (e.g., over a network), and may also include one or more user I/O interfaces 760, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described herein can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited herein.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the functions of one or more embodiments discussed herein. The computer-readable medium may be transportable such that the program stored thereon can be loaded onto any computing device to implement aspects of the techniques discussed herein. In addition, it should be appreciated that the reference to a computer program which, when executed, performs any of the functions discussed herein, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description, but is not intended to be exhaustive or to limit the implementations to the precise form disclosed. Modifications and variations are possible in light of the teachings herein or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described herein, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

V. Exemplary Tests for Use with the Downloadable Software Application and Software Ecosystem

The downloadable software application can provide instructions to administer a test and/or process test results for any type of test. Described herein are examples of tests that can be used with the downloadable software application. In some embodiments, the tests described herein are end-point nucleic acid amplification tests.

A. Test Process

The test process generally includes obtaining a sample, processing the sample, and analyzing the sample. The sample, in some embodiments, is saliva or a nasal swab. Sample processing can occur in a number of different manners, but results in lysing the sample and amplifying the nucleic acids. Analysis of the sample, e.g., determination of whether the sample is positive or negative for one or more viral illnesses, may comprise the use of a readout device. In some embodiments, the readout device comprises a lateral flow strip. The lateral flow strip, in some embodiments, comprises a control location (“control line”) and at least one test location (“test line”). Each test location is associated with a particular viral illness, e.g., COVID19, influenza type A or influenza type B, or other. Multiple test locations on a test strip, each associated with a different illness are envisioned as well. In some embodiments, each of the steps of the test is guided by a downloadable software application as described herein (e.g., a companion mobile application (“app”), for example, on a cellular phone (e.g., smartphone)).

1. Sample Collection

The test includes using a collection component (e.g., a swab or pad) to collect a sample. Exemplary samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. After the sample has been collected, the swab may be added to a sample tube, and a buffer, such as phosphate-buffered saline (PBS) may be added to the tube.

2. Lysis of Sample

In some embodiments, UD and an RNAse inhibitor may be added subsequently. Lysis and RNA extraction can be performed using any methods known in the art. In some embodiments, lysis is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. Non-limiting examples of suitable enzymes include lysozyme, lysostaphin, zymolase, cellulose, protease, and glycanase. In some embodiments, the one or more lysis reagents comprise one or more detergents. In some embodiments, lysis is accomplished with a lyophilized lysis pellet. In some embodiments, cell lysis is performed at room temperature (e.g., 20° C.-22° C.). In still other embodiments, cell lysis is accomplished by applying heat to a sample (thermal lysis).

3. Amplification of Sample

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. As described herein, the nucleic acid amplification reagents can be loop-mediated isothermal amplification (LAMP) reagents, recombinase polymerase amplification (RPA) agents, and/or agents for NEAR reactions. In some embodiments, the loop-mediated isothermal amplification (LAMP) protocol or the recombinase polymerase amplification (RPA) protocol described below includes a modified nucleotide, for example, deoxyuridine triphosphate (dUTP), during amplification. In some embodiments, the one or more reagents comprise one or more reagents for CRISPR/Cas detection.

In some embodiments, the one or more reverse transcription reagents comprise a reverse transcriptase, a DNA-dependent polymerase, and/or a ribonuclease (RNase). A reverse transcriptase generally refers to an enzyme that transcribes single-stranded RNA (ssRNA) into complementary DNA (cDNA) by polymerizing deoxyribonucleotide triphosphates (dNTPs). An RNase generally refers to an enzyme that catalyzes the degradation of RNA. In some cases, an RNase may be used to digest RNA from an RNA-DNA hybrid. In some an embodiments, a reverse transcriptase and a DNA-dependent polymerase are used. Reverse transcriptases (also known as RNA-dependent DNA polymerases), are enzymes having a DNA polymerase activity that transcribe single-stranded RNA (ssRNA) into a complementary single stranded DNA (cDNA) by polymerizing deoxyribonucleotide triphosphates (dNTPs). In some embodiments, RNAse may be used to digest the RNA away from an RNA-DNA hybrid. RNAses are commercially available (e.g., from ThermoFisher Scientific, New England BioLabs, etc.). In some embodiments, the one or more reagents comprise an RNase inhibitor (e.g., a murine RNase inhibitor). In some embodiments, the one or more reagents comprise one or more nucleic acid amplification reagents.

In any of the embodiments described herein, the reagents, including the LAMP reagents or the RPA reagents, may be lyophilized and formulated as one or more beads. These beads are referred to herein as “amplification beads” or “amplification pellets.” As described herein, the amplification beads may be added to any of the tests provided herein, for example, as part of a cap/lid designed to release the amplification bead(s) into solution after the sample has been mixed into a buffer or as part of a blister pack in a lid, such that the amplification bead is contacted with the sample.

a. Recombinase Polymerase Amplification (RPA)

In some embodiments, the reverse transcription step is followed by recombinase polymerase amplification (RPA) in order to amplify the resulting DNA. RPA is an isothermal amplification technique that allows for fast, portable and extremely sensitive nucleic acid detection. RPA is a quick reaction (results are typically generated within 10 minutes) and does not require extensive instrumentation and/or reagents, making it well-suited for point-of-care use in settings with minimal resources. Following amplification, RPA products generated by the reaction can be released from the reaction test tube onto the sample portion of a lateral flow (LF) strip, as described herein. Depending on whether or not the target nucleic acid was detected, visible colored lines form on the strip. In this manner, RPA-LF can be used as the basis for the at-home nucleic acid tests described herein. For example, RPA-LF may be used for diagnosing or aiding in the diagnosis of infection, such as diagnosing COVID-19 or other diseases.

RPA is known in the art, and typically includes a recombinase agent, which is contacted with a forward and a reverse nucleic acid primer to form a first and a second nucleoprotein primer. In some embodiments, the RPA reagents comprise a probe, a forward primer, and a reverse primer. The probe, forward primer, and reverse primer may be designed for each target nucleic acid a diagnostic device is configured to detect. In some embodiments, the RPA reagents comprise one or more recombinase enzymes. In some embodiments, the RPA reagents comprise one or more single-stranded DNA binding proteins. In some embodiments, the RPA agents comprise a DNA polymerase. In some embodiments, the RPA agents comprise an endonuclease. In some embodiments, the RPA reagents comprise dNTPs (e.g., dATP, dGTP, dCTP, dTTP).

b. Loop-Mediated Isothermal Amplification (LAMP)

In some embodiments, the DNA sample is subjected to loop-mediated isothermal amplification (LAMP) instead of RPA. In some embodiments, the LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

In some embodiments, the LAMP reagents comprise a FIP and a BIP for one or more target nucleic acids. In some embodiments, the LAMP reagents comprise an F3 primer and a B3 primer for one or more target nucleic acids. In some embodiments, the LAMP reagents comprise a forward loop primer and a backward loop primer for one or more target nucleic acids. In some embodiments, the control nucleic acid is a nucleic acid sequence encoding human RNase P. In some embodiments, one or more LAMP primers comprise a label. In some embodiments, the LAMP reagents comprise a DNA polymerase with high strand displacement activity. In some embodiments, the LAMP reagents comprise deoxyribonucleotide triphosphates (“dNTPs”). In some embodiments, the LAMP reagents comprise magnesium sulfate (MgSO4). In some embodiments, the LAMP reagents comprise betaine.

For example, a biotinylated FIP primer is incubated with the nucleic acid sample (e.g., DNA) for 30 minutes at 65° C. Then, a specific FITC-labeled probe is added to the reaction mixture and incubated for another 10 minutes at 65° C., resulting in a dual-labeled LAMP product. Then, detection buffer containing rabbit anti-FITC antibodies coupled to colloidal gold is mixed with the reaction mixture, and the lateral flow test strip is inserted into the tube. In a positive reaction, the double labeled LAMP product migrates with the buffer flow and is retained at the test line by a biotin ligand present on the test line. The gold coupled anti-FITC antibody binds to the FITC molecule at the probe, and an opaque band develops over time. In a negative sample, the reactions do not occur, and no opaque band develops in the test line. The control line comprises an anti-rabbit antibody, which retains some of the unbound gold-conjugated antibody, resulting in an opaque band in the control line.

In one embodiment, the nucleic sample is subjected to colorimetric LAMP. A gold- or antigen-labeled probe is added to the sample. If the probes bind their target, then the labeled probes are dispersed throughout the solution during the reaction (resulting in one color). If, however, the probes do not bind their target, they aggregate instead, resulting in a second color. By reading the different colors of the test, a user can determine whether a sample is positive or negative for a target sequence (e.g., COVID19).

c. NEAR In some embodiments, the nucleic acid amplification reagents are NEAR reagents.

NEAR generally refers to a method for amplifying a target nucleic acid using a nicking endonuclease and a strand displacing DNA polymerase. In some cases, NEAR may allow for amplification of very small amplicons. In some embodiments, the NEAR reagents comprise a forward primer. In some embodiments, the NEAR reagents comprise a DNA polymerase. In some embodiments, the nucleic acid amplification reagents are tHDA reagents. In some embodiments, the tHDA reagents comprise a forward primer and a reverse primer. In some embodiments, the tHDA reagents further comprise a probe.

4. Detection

The processed RNA sample is then detected using any means known in the art. In some embodiments, detection involves a lateral flow test. In other embodiments, detection involves a colorimetric assay.

Lateral flow tests or assays, comprise a test strip comprising, in order of flow direction a sample region and a results region. The processed sample (e.g., a saliva sample which has undergone amplification using any one of the methods described above or known in the art), is added to the sample region. The results region comprises at least one test line and a control line. The test line comprises a probe, for example, an antibody, that recognizes a target a sequence. In some embodiments, the target sequence is a sequence from the processed viral DNA (e.g., a coronavirus- or influenza-specific DNA sequence). Thus, in some embodiments, the sample interacts with the test line if the sample comprises the target sequence and is detectable, and, in other embodiments, the sample does not interact with the test line because the sample does not comprise the target sequence (and therefore, is not detectable). In some embodiments, the target sequence is specific for SARS-CoV-2. In some embodiments, the target sequence is specific for influenza type A or influenza type B. In some embodiments, the test region comprises more than one test line. For example, the test region may comprise a first test line that is specific for coronavirus (e.g., COVID19) and a second test line that is specific for influenza (e.g., influenza type A or influenza type B).

In one embodiment, following amplification, products (control, and if present, test) generated by the reaction are released from the sample tube onto the sample pad of a lateral flow dipstick. By passive capillary flow, products in the sample are wicked over the conjugate pad where a visible dye attaches to the products. As the labeled amplicons migrate across the dipstick they pass over multiple discrete bands of immobilized antibodies. The antibodies in a given band will capture one of the amplified products (control or test) with high specificity. In this fashion, control products are captured on one band, test products are captured on another. When the products are captured on their respective bands, the dye attached to each product generates a colored line on the dipstick. The presence of a visible Positive Control band indicates that the test ran successfully, while the presence of the test band indicates the target analyte was detected in the patient's sample (in this case, COVID-19). In this manner, SARS-CoV-2 infection and confirmation of proper test function is indicated by the appearance or absence of a colored visible band at the appropriate physical locations along the lateral flow strip. An exemplary lateral flow strip key is provided in FIG. 7.

In some embodiments, the detection is performed through a colorimetric assay, that is, a chromogenic reaction is performed. For example, the processed sample is exposed to reagent that undergoes a color change when bound to the viral DNA, such as with an enzyme-linked immunoassay. In some embodiments, the assay further comprises a stop reagent, such as sulfonic acid. That is, when the processed sample is mixed with the reagents, the solution turns a specific color (e.g., red) if the pathogenic DNA is present, and the sample is positive for the virus. If the solution turns a different color (e.g., green), the pathogenic DNA is not present and the sample is negative for the virus. As described above, the colorimetric assay may be a colorimetric LAMP assay; that is, the LAMP reagents react in the presence or absence of a target sequence (e.g., from COVID19) to turn one of two colors.

5. Analysis/Diagnosis

The results, in some embodiments, are determined with the aid of a software-based application. The application can be downloaded to a smartphone or device, and then guides a user through steps to use the test kit or strip. The user, in some embodiments, is the person (or people) performing the test. The application, in further embodiments, may validate that the test was performed correctly. That application also can be used to report results and maintain data. The application can communicate results to a central database, a doctor, or other. For example, in one embodiment, if the test is valid and shows a positive or negative result, that information may be reported to the user (via the mobile app) and to the CDC via a cloud system.

In one embodiment, a user inputs demographic and symptom information into the mobile app, and then proceeds through the test instructions. The application also may be used to receive and process test results. In addition, the application may receive and process serological antibody or antigen test results.

The test, in some embodiments, may be used to diagnose at least one disease or disorder caused by a pathogen, as described herein. In some embodiments, the tests may be designed so that a user can differentiate between one or more diseases or disorders (e.g., a lateral flow test comprises more than one test line). In one embodiment, the lateral flow test comprises a test line for SARS-CoV-2 and a test line for an influenza (e.g., Type A or Type B). In another embodiment, the lateral flow test comprises a test line for SARS-CoV-2, influenza Type A, and influenza Type B. In further embodiments, the test may be used to differentiate between viral and bacterial infections. In some embodiments, a diagnostic device is configured to detect a first target nucleic acid. In some cases, the first target nucleic acid is a nucleic acid of a pathogen. The pathogen may be a viral, bacterial, fungal, protozoan, parasitic, or other pathogen.

B. Cartridge Embodiments

In some embodiments, one or more of processing, detecting, or analyzing steps are performed with a cartridge. In some embodiments, a sample is processed using the various reservoirs or chambers of the cartridge, each of which are interconnected via channels molded into cartridge plastic. In some embodiments, a silicone peristaltic layer forms “pump lanes” associated with various channel connections, which by action of pumping with a user-operated roller pumping tool, drives sample and reagent between reservoirs at the appropriate times. Passive valves in each pump lane isolate the reservoirs during non-pumping events. Heat (e.g., via a PCB heater) is applied to the underside during lysis and amplification.

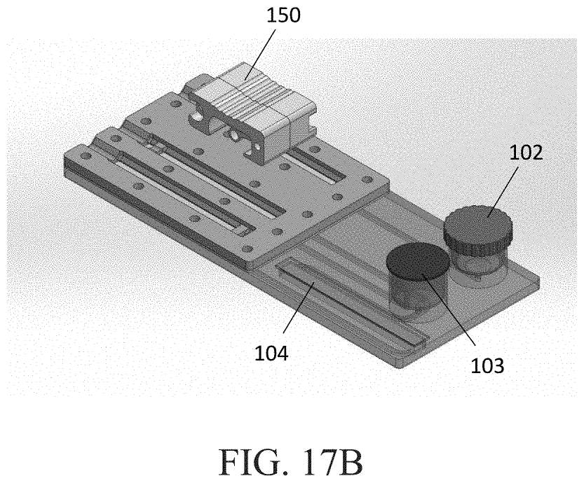

An exemplary cartridge is shown in FIG. 17A. In FIG. 17A, 100 labels the pumping tool, 101 is a seal plate, 102 is a lysis reservoir (chamber), 103 is an amplification reservoir (chamber), 104 is a readout strip, 105 is a bottom seal layer, 106 is a molded cartridge, and 107 indicates a peristatic membrane. The lysis reservoir, in some embodiments, is pre-loaded with solution, such that the user removes the cap and punctures the seal at the base of the reservoir, using a sterile puncture tool provided with the cartridge. The user then adds the sample to the lysis reservoir by swirling the sample-containing swab around in the reservoir to mix it with the solution. The swab is then removed, and the cap is loosely placed back on the reservoir. The reservoir is then heated to an appropriate temperature (e.g., 65C) to allow the sample to lyse (e.g., thermal lysis is performed). In other embodiments, the lysis reservoir comprises enzymes and/or detergents, and lysis is performed at room temperature. The lysed sample is then moved to the amplification reservoir (103) using pump lane (channel) 1. The amplification reservoir, in some embodiments, comprises lyophilized amplification reagents (e.g., a lyophilized amplification bead), as described herein. The amplification strategy may be RT/RPA or LAMP. Some of its ambient air may be pumped out using pump lane (channel) 3. Then, the lysate from the lysis reservoir (102) is added to the amplification reservoir, where it interacts with the lyophilized amplification reagents (e.g., in some embodiments, an amplification bead, as described herein). In some embodiments, the resulting solution is heated (e.g., at 37 C) for amplification to occur. After amplification, the resulting solution is then transported to the readout strip (lateral flow strip) (104) using pump lane (channel) 4. The assay is completed, and the user may determine the results using any of the methods described herein (e.g., comparing the results to a key, using a mobile app, etc.). In some embodiments, the pumping tool wraps around the cartridge (e.g., as shown by pumping tool 100 in FIG. 17A). In other embodiments, the pumping tool sits above the cartridge (e.g., as shown by pumping tool 150 in FIG. 17B), so that the user can slide it forward along the specified pump lane. The pumping tool, in these embodiments, may comprise three molded parts, two of which are two copies of the body rotated 180 degrees with respect to each other. The third is the roller itself. In some embodiments, the assembly is designed to snap or press together without additional fasteners, and in other embodiments, additional fasteners (e.g., screws) are used to secure the pumping tool.

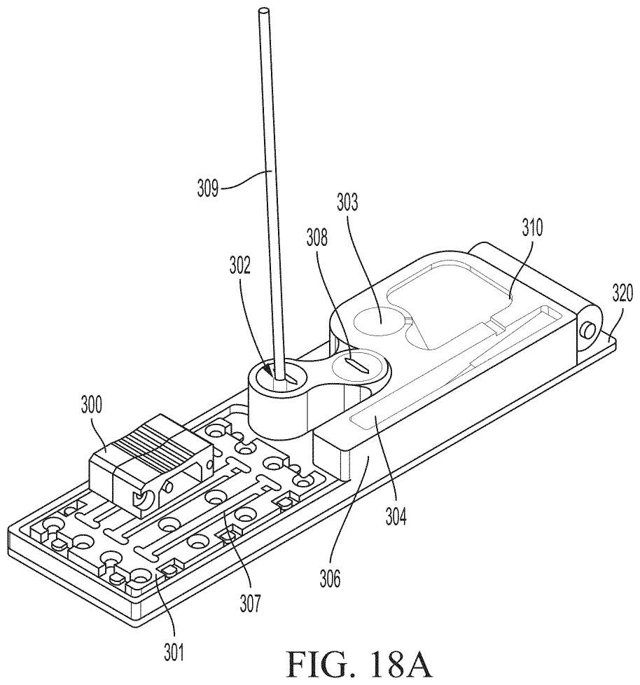

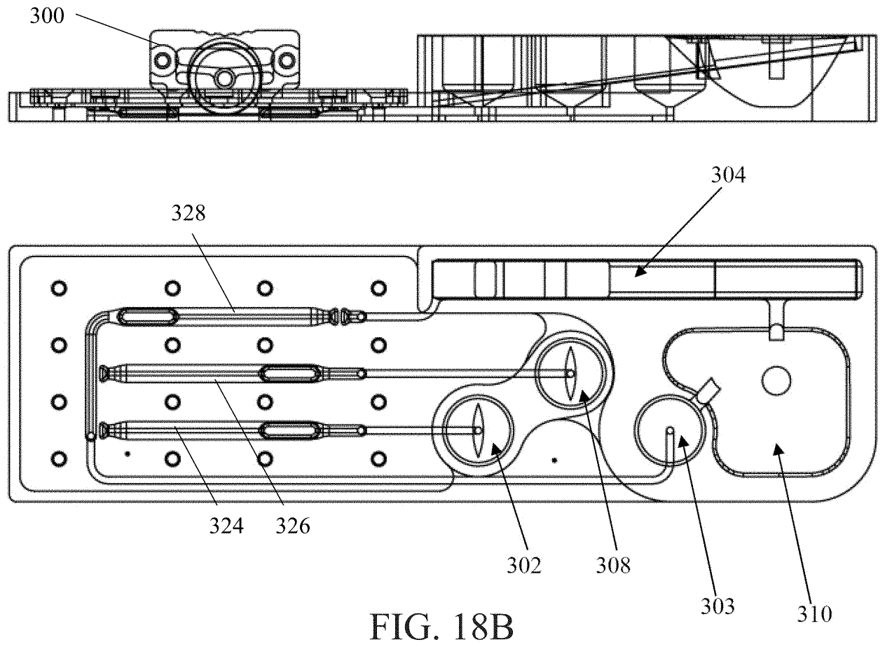

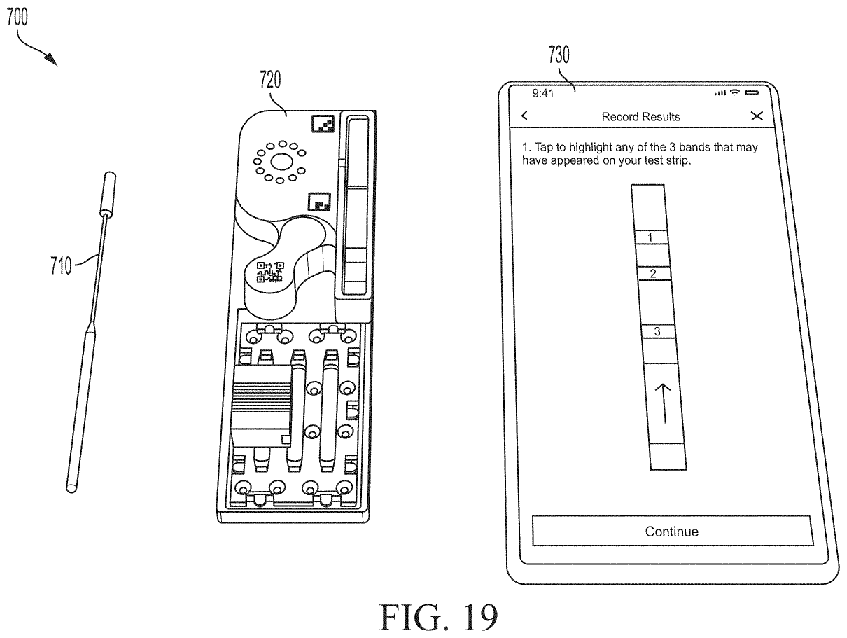

In FIG. 18A, this embodiment comprises a dilution buffer reservoir (308), a swab (309), and an air expansion reservoir (310), in addition the components described above: 300 shows the pumping tool, 301 is a seal plate, 302 is a lysis reservoir, 303 is an amplification reservoir, 304 is a readout strip, 306 is the molded cartridge, and 307 indicates a peristatic membrane. As is depicted in FIG. 18A, 309 is a swab. In some embodiments, the swab (309) is inserted into punctured film of the lysis reservoir (302) to deposit the sample into the lysis solution. The lysate (e.g., after optional heating) is then moved to the amplification reservoir (303). In some embodiments, the lysate is transported using a pumping tool (300). The user slides the pumping tool back and forth in the appropriate pump lane (channel) in sequential order, as described by the provided instructions (e.g., a mobile application). FIG. 18B illustrates the different channels, as seen through the bottom (backside) of the cartridge (lane 1 (324) is depicted at the bottom of the figure). Lanes 1 (324) and 2 (326) serve as inputs to a common channel (connected to the amplification reservoir) and lane 3 (328) serves as output to read-out strip.

The amplification reservoir (chamber) (303) comprises lyophilized amplification reagents (e.g., a lyophilized amplification bead), as described herein. After amplification, the resulting sample is transported to the readout strip (304) using a unique pump lane. The readout strip, in some embodiments, uses angled pocket geometry. The assay is completed, and the user may determine the results using any of the methods described herein (e.g., comparing the results to a key, using a mobile app, etc.). The cartridge also includes an air expansion reservoir (310) which maintains the atmospheric pressure in the amplification reservoir and readout strip area, while maintaining a hermetic seal to prevent contamination. A thin heat-seal plastic film layer behaves as a chemical barrier and as a pressure diaphragm. The peristaltic membrane (307), in some embodiments, comprises die-cut silicone. Likewise, the seal plate (301), in some embodiments, comprises FR4/G10, and may be attached to the main-body cartridge (311) by fasteners (e.g., glue, screws). If a dilution buffer is not needed, its corresponding lane can be blocked off and is not functional (e.g., to prevent user error).

In some embodiments, the cartridge further comprises a printed circuit board (PCB) heater and battery. In some embodiments, the heater is controlled by a mobile application. In other embodiments, the companion mobile application alerts a user as to when the heating protocol (e.g., lysis, amplification) is complete. In some embodiments, the mobile application is able to store information regarding the temperatures used during the processing steps. In a further embodiment, the heating device is connected to the mobile application via a wired connection or through a wireless connection such as Bluetooth®. In some embodiments, the wireless, e.g., Bluetooth®, connection allows the mobile application to store all of the information from the heating device. In some embodiments, the wireless, e.g., Bluetooth®, connection allows a user to select the different heating/cooling protocol as needed. In some embodiments, the heating/cooling protocol may be selected remotely (e.g., not by the immediate user).

C. Blister Pack Embodiments

In some embodiments, the different reagents are stored in lab on chip reagent blister packs. In some embodiments, the blister packs are multi-chamber blister packs; that is, the blister pack may store multiple components (both liquid and solid) in different chambers. For example, lyophilized reagents can be stored in individual chambers, while the buffers or solutions necessary to resuspend the lyophilized reagents can each be stored in separate chambers, separated by a frangible seal. In one embodiment, the delivery of each reagent is fully automated. For example, the user inserts a sample to a sample collection region of the blister pack and then activates the blister pack, which supplies all of the reagents in the correct amount and at the appropriate time, such that the sample is processed as described herein. The blister pack, in some embodiments, further comprises a readout component, wherein the user is alerted as to whether the sample was positive or negative for the tested viral illness (e.g., coronavirus or influenza).

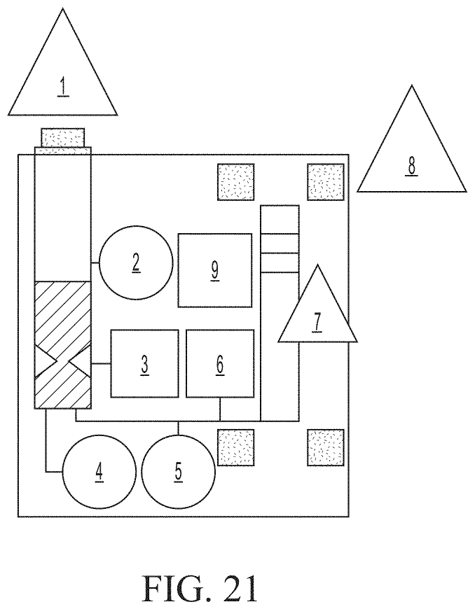

An exemplary blister pack setup is depicted in FIG. 21. The tube contains the sterilization buffer. A sample is added through the sample port (1). A blister pack (2) comprising UDG and other enzymes necessary for lysis is released into the tube. In some embodiments, the region also comprises a heating element for lysis. A mechanism to downselect the sample volume, if necessary, through physical means is also provided (3). This is followed by an amplification blister pack (4) and an optional dilution blister pack (5). The sample is then flowed across a lateral flow strip (7), with mechanisms in place to ensure the sample accesses the flow strip at the appropriate time (e.g., after the processing is complete) (6).

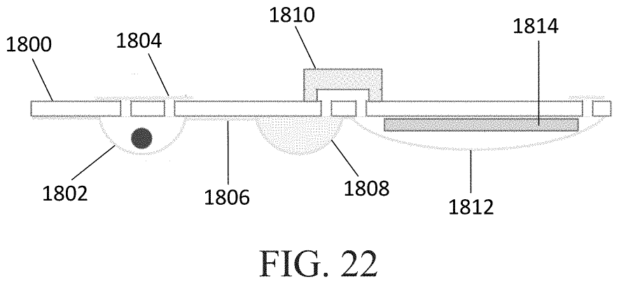

In FIG. 22, the blister pack 1800 may comprise a first chamber 1802, a sample port 1804, a seal 1806, a second chamber 1808, a valve 1810, a third chamber 1812, and a lateral flow assay strip 1814. As described herein in some examples, the first chamber 1802 may comprise one or more amplification reagents (e.g., LAMP, RPA, NEAR reagents) in solid form (e.g., lyophilized). The second chamber 1808 may comprise a dilution buffer. The third chamber 1812 may comprise the lateral flow assay strip 1814.

In some embodiments, the swab is mixed with the sample buffer and a lyophilized lysis mix is added when a frangible seal is broken. In some embodiments, heat lysis is used. That is, the sample is added to the sample buffer and then heat is applied to lyse the sample. The sample is then moved to a lyophilized amplification mix chamber (blister) comprising the for amplification. Similarly, a dilution buffer is added to the lyophilized mixture when its frangible seal is broken. The sample, after processing, is then added to a lateral flow device to be analyzed. In some embodiments, the results on the lateral flow strip are interpreted using a mobile software based application, downloadable to a smart device, such as that described herein.

D. Diagnostic Test Swabs

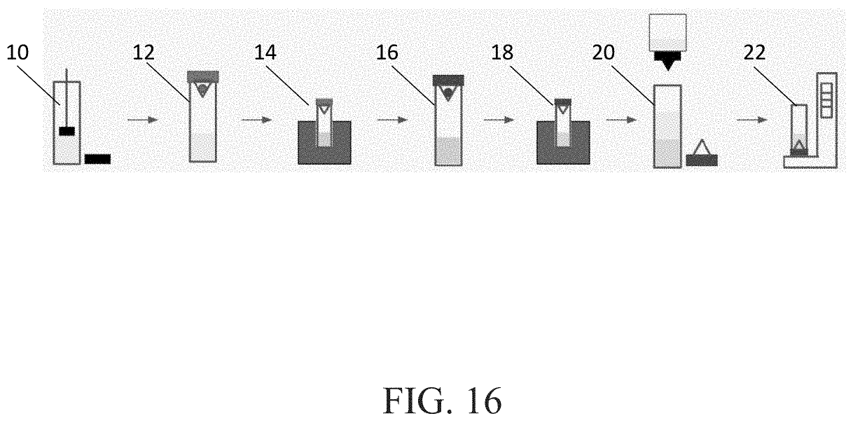

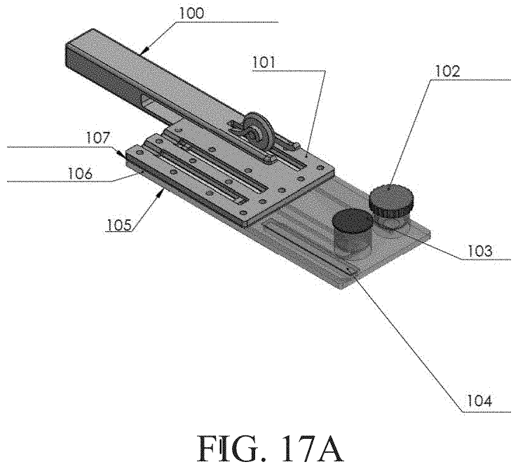

The testing procedure may be contained within a single-use diagnostic test swab, as depicted in FIG. 16. Briefly, a sample (e.g., anterior nares sample) may be taken using the foam swab end of the device. The sample may then be immersed in a rehydration buffer (step 10), a lysis cap can be placed on the tube and the tube can be inverted until dissolved (step 12) and the sample can be heated to any relevant temperature (step 14) such as 65C before preceding through the remainder of the testing process. The cap can be swapped for an amplification cap, and the tube can be inverted until dissolved (step 16). The sample cam be heated to any relevant temperature (step 18) such as 65C. A dilution buffer can be added to the tube (step 20), and the tube can be added to a readout device (step 22), where the test result can be read out after an appropriate amount of time such as 10 minutes.

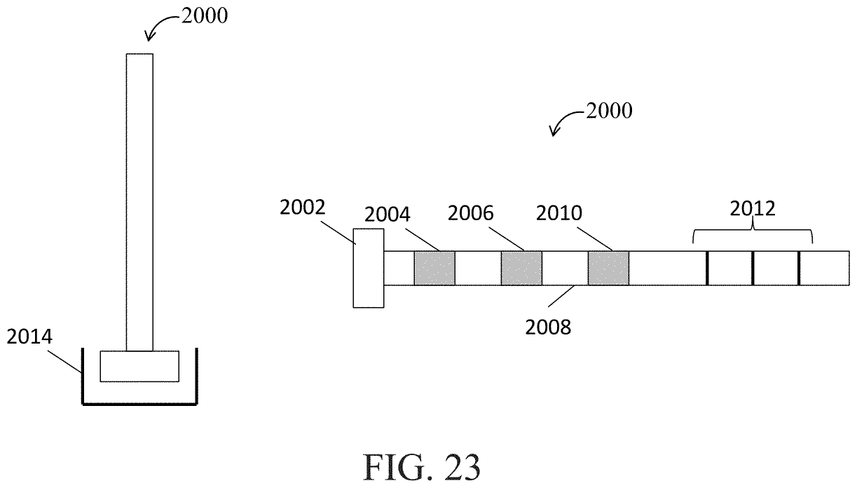

As shown in FIG. 23, the swab 2000 can be placed into a heated buffer 2014. The sample then moves through the handle 2002 of the swab 2000 to a lysis region 2004. The lysis region 2004 may comprise lysis buffer, in addition to lysis enzymes and detergents. In some embodiments, the lysis buffer is contained in a blister pack, which is punctured to release the buffer at the appropriate time. In some embodiments, the lysis region is a lysis lateral flow strip; that is, the sample proceeds through the region capillary action and interacts with lysis reagents impregnated in the flow strip. After the sample has been lysed, it moves via capillary action, to the amplification region 2006. The sample is slowed, if necessary, by materials in the lateral flow strip (2008), in order to ensure proper and sufficient amplification so that the processed sample will be able to be analyzed. An antibody region 2010 can be included on the swab 2000. The processed sample the progresses to the readout region of the test, which comprises a results lateral flow test strip 2012. In some embodiments, the amplification lateral flow strip and the results lateral flow strip are one continuous flow strip; that is, there is an amplification region and a results region on a single lateral flow strip.

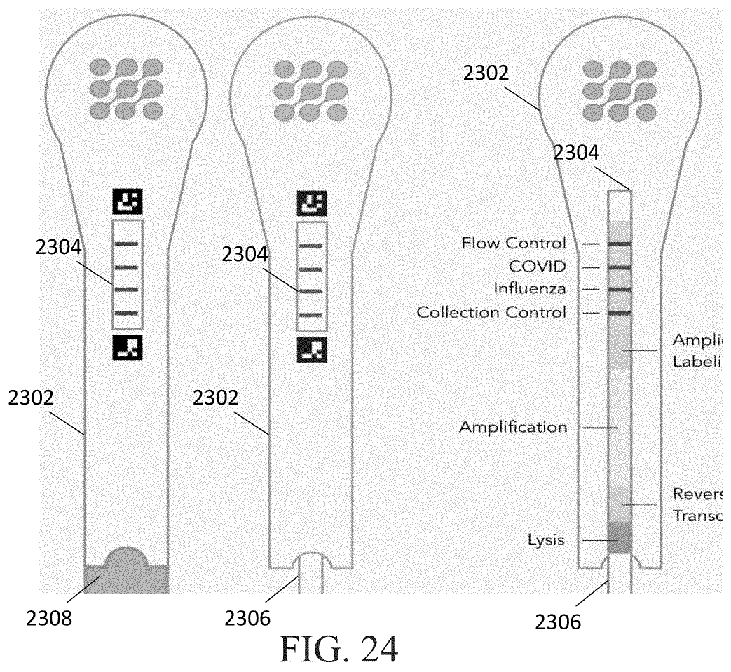

The sample collection swab may be a separate swab or in an embodiment built into the test device itself, as shown in FIG. 24. Shown in the embodiment of FIG. 24 is an all-in-one test device including tube 2302, lateral strip 2304, and sample collection swab 2306. A removable cover 2308 when removed reveals the swab 2306. Also included is a stand for the tube 2302 (not shown).

E. Further Exemplary Diagnostic Devices

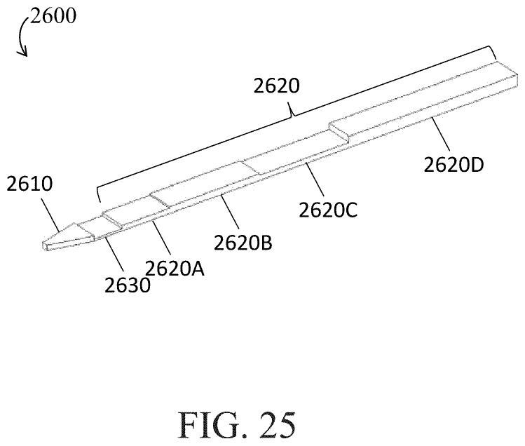

In some embodiments, a diagnostic device comprises an outer casing and an inner member that is movable within the outer casing. In certain embodiments, the diagnostic device comprises a sample-collecting component that is coupled to the outer casing and/or the inner member. In some embodiments, a diagnostic device comprises a substrate. An exemplary substrate is shown in FIG. 25. In certain embodiments, the substrate comprises a reagent delivery region and a lateral flow assay region. For example, in FIG. 25, substrate 2600 comprises reagent delivery region 2610 and lateral flow assay region 2620, which comprises sample pad 2620A (e.g., where a liquid sample is introduced to lateral flow assay region 2620), particle conjugate pad 2620B (e.g., where labeled nanoparticles may be located), test pad 2620C (e.g., where the one or more test lines and/or control lines may be located), and wicking area 2620D. In FIG. 25, substrate 2600 comprises separation region 2630 positioned between reagent delivery region 2610 and assay region 2620.

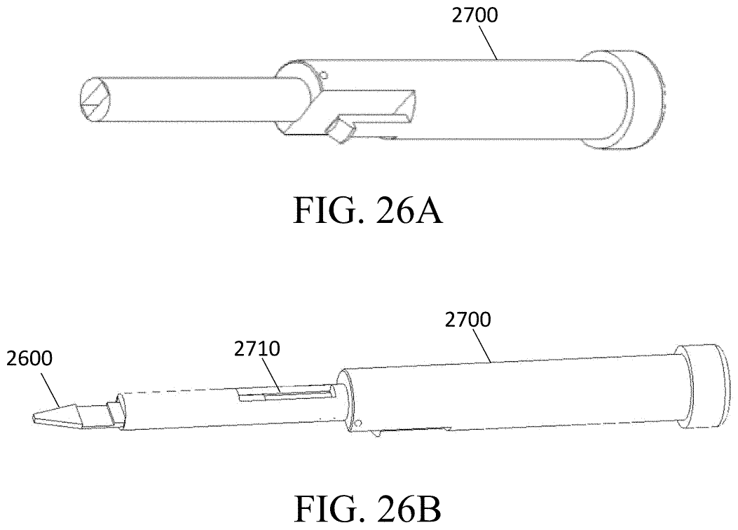

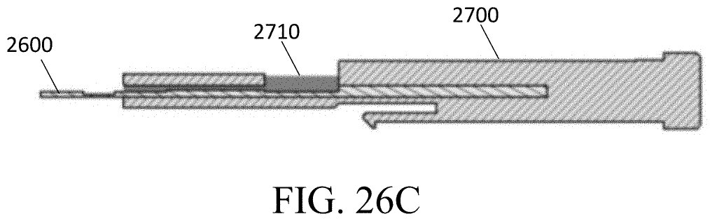

In some embodiments, a substrate may be associated with an inner component. As one example, FIGS. 26A-26C show an exemplary inner component associated with a substrate, according to some embodiments. FIG. 26A shows an external view of inner component 2700, and FIG. 26B shows an external view of substrate 2600 positioned within inner component 2700. As shown in FIG. 26B, inner component 2700 may comprise an opening 2710 through which at least a portion of substrate 2600 is visible. FIG. 26C shows a cross-sectional view of substrate 2600 positioned within inner component 2700.

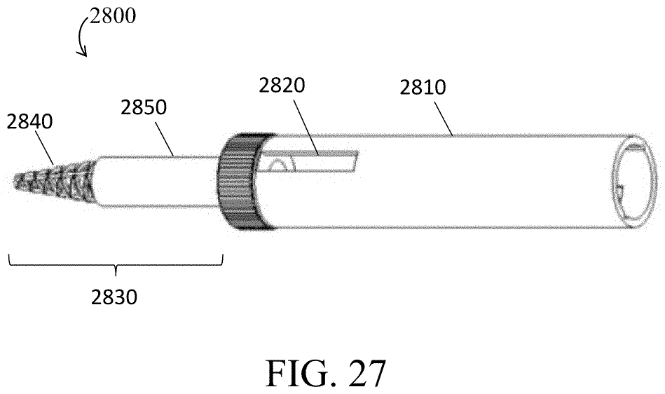

In some embodiments, a substrate and an inner component may be associated with an outer component. An illustrative embodiment of an outer component is shown in FIG. 27. In FIG. 27, outer component 2800 comprises outer casing 2810, which has an opening 2820. Like inner component 2700, outer casing 2810 may be formed from any suitable material. In some embodiments, outer casing 2810 comprises a thermoplastic polymer and/or a metal. Outer casing 2810 may be formed by injection molding, an additive manufacturing process (e.g., 3D printing), and/or a subtractive manufacturing process (e.g., laser cutting). In some embodiments, outer component 2800 further comprises sample-collecting component 2830. A sample-collecting component may be removably or permanently coupled to either an outer component or an inner component of a diagnostic device. In FIG. 27, sample-collecting component 2830 comprises swab element 2840 and stem element 2850.

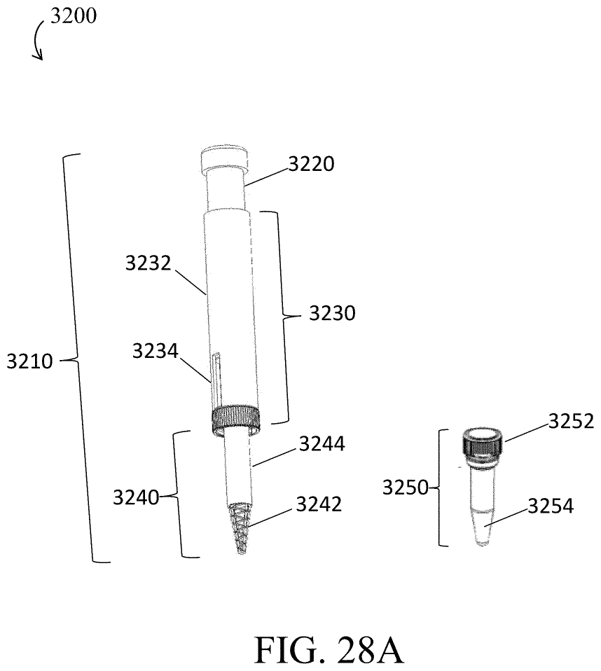

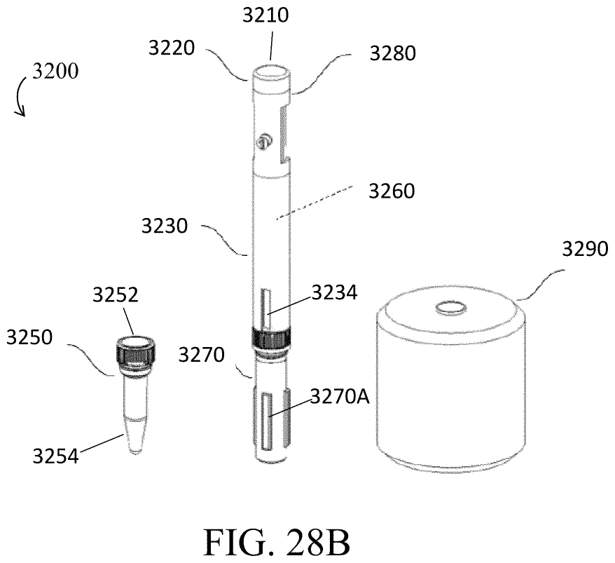

Some embodiments are directed to a diagnostic test kit. An illustrative embodiment of a diagnostic test kit is shown in FIG. 28A. In FIG. 28A, diagnostic test kit 3200 comprises diagnostic device 3210 and reaction tube 3250. Diagnostic device 3210 comprises inner component 3220, outer component 3230, and sample-collecting component 3240. Outer component 3230 comprises outer casing 3232 and opening 3234 in outer casing 3232. Sample-collecting component 3240 comprises swab element 3242 and stem element 3244. In addition to diagnostic device 3210, diagnostic test kit 3200 further comprises reaction tube 3250. As shown in FIG. 28A, reaction tube 3250 comprises cap 3252 and fluidic contents 3254. In some embodiments, fluidic contents 3254 comprise a reaction buffer.

In operation, cap 3252 of reaction tube 3250 may be removed, exposing fluidic contents 3254. In some embodiments, sample-collecting component 3240 is used to collect a sample (e.g., nasal secretion, oral secretion, cell scraping, blood, urine) from a subject (e.g., a human subject, an animal subject). In some instances, for example, swab element 3242 is inserted into a nasal or oral cavity of the subject to collect the sample. Sample-collecting component 3240, bearing the sample, is then inserted into fluidic contents 3254 of reaction tube 3250. In some embodiments, outer component 3230 may be secured to reaction tube 3250 (e.g., by a screw, a snap locking mechanism, or other fastener). A first action (e.g., pushing inner component 3220 into outer component 3230, rotating inner component 3220 relative to outer component 3230) is performed that moves inner component 3220 relative to outer component 3230 such that a first portion of inner component 3220 is in physical contact with fluidic contents 3254 of reaction tube 3250. In some cases, a second action (e.g., pushing inner component 3220 into outer component 3230, rotating inner component 3220 relative to outer component 3230) is performed that further moves inner component 3220 relative to outer component 3230 such that a second portion of inner component 3220 is in physical contact with fluidic contents 3254 of reaction tube 3250. In some cases, an indicator of the presence or absence of a target nucleic acid may be detectable through opening 3234 of outer casing 3232.

In some embodiments, a diagnostic test kit further comprises a heating unit. In FIG. 28B, diagnostic test kit 3200 comprises diagnostic device 3210, reaction tube 3250, and heating unit 3290. In some embodiments, heating unit 3290 heats fluidic contents 3254 of reaction tube 3250 to one or more desired temperatures for a desired amount of time.

F. Kits

Any of the tests described herein may formulated as a kit. As used herein a “kit” comprises a package or an assembly including one or more of the test compositions (e.g., any number of reaction tubes, wells, chambers, or other vessels).

A kit may, in some cases, include instructions in any form that are provided in connection with the compositions of the invention in such a manner that one of ordinary skill in the art would recognize that the instructions are to be associated with the compositions of the invention. The instructions may include instructions for performing any one of the tests provided herein. The instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications). In some embodiments, the instructions are provided as part of a software-based application, as described herein. Several exemplary kits and methods of using them are described below.

FIG. 19 illustrates an exemplary test kit 700 including sample collecting swab 710, test cartridge 720 and smart phone or device 730 for displaying results. The smart device 730 may include a downloadable software application which can provide instructions for use, a result readout, and/or communication platform. The smart device 730 may be connected wirelessly and communicate with the test cartridge 720.

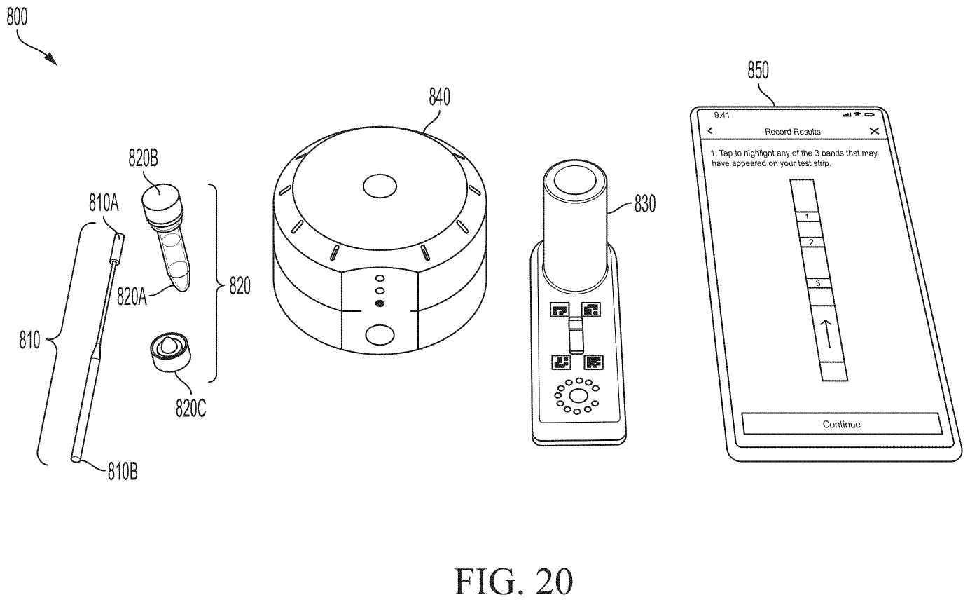

FIG. 20 illustrates elements of a test kit 800 according to an embodiment. As shown, the kit 800 includes a sample collecting swab 810 with a swab element 810A and a stem element 810B, test tube with caps 820, holder 840 (e.g., a heater), cartridge 830 and smart phone 850. In certain embodiments, the reaction tube 820 may comprise a vial or tube 820A, a first cap 820B, and a second cap 820C. As shown in FIG. 20, the first cap 820B and/or the second cap 820C may be screw-top caps or any other type(s) of removable caps. In certain embodiments, the second cap 820C may comprise one or more reagents (e.g., lysis reagents, nucleic acid amplification reagents, CRISPR/Cas detection reagents, and the like). The fluidic contents of the reaction tube 820 may comprise a reaction buffer. The tube 820A of the reaction tube 820 may be sized to contain or hold any suitable volume of the reaction buffer.

The kit may comprise collecting a sample on a swab (e.g., a foam swab) and then lysing the sample using any one of the methods provided herein (e.g., thermal or enzymatic lysis with a lysis buffer). In some embodiments, the lysis reagents may be present on an absorbent pad, and a blister pack comprising a rehydration buffer may be broken, rehydrating the lysis reagents. The sample may then be added to the lysis solution. In some embodiments, a heating element is present below the lysis setup of the reaction, and lysis occurs at a specific temperature, e.g., 65C. After lysis, the resulting lysate is added to an amplification lateral flow strip. The amplification lateral flow strip comprises the reagents necessary for RT/RPA or LAMP. When the lysate enters the amplification lateral flow strip, it rehydrates the amplification reagents, permitting the sample to be amplified. In some embodiments, the amplification lateral flow strip further comprises a blister pack comprising a dilution buffer. The dilution buffer may then be released to the lateral flow strip, to rehydrate the reagents and to dilute the lysate, by piercing the blister pack. In other embodiments, the amplification step is accomplished with an absorbent pad comprising lyophilized amplification reagents (e.g., LAMP reagents or RT/RPA reagents) in sequential order (e.g., in RT/RPA, reverse transcriptase reagents followed by RPA reagents). Similarly, a dilution buffer may be released from a blister pack to rehydrate the reagents. In some embodiments, the amplification lateral flow strip comprises a heating element (e.g., an element capable of heating the lateral flow strip comprising the amplification reagents). After amplification, the sample is moved to a results lateral flow strip, wherein the sample is queried for a sample positive (human) control, a test control, COVID19, and influenza (type A and/or type B) using any of the methods described herein. In some embodiments, the results are reported through a mobile application described herein.

In one embodiment, an isothermal colorimetric LAMP kit is provided. The kit comprises at least a heat source and a cartridge, although the heat source may be integrated into the cartridge. A sample is deposited in the centrally-located sample port, where it is combined with a buffer, such as a lysis buffer. The sample is heated to lyse the cells. Then, the sample flows from the sample port to the four peripheral chambers. Each peripheral chamber comprises LAMP reagents, including a unique set of primers (e.g., primers specific for a positive test control, primers specific a negative test control, primers specific for a positive sample control, and primers specific for the tested virus). When the sample reaches each of the peripheral chambers, a colorimetric reaction occurs. The results are visible through the clear covering of each peripheral chamber. As described below, the results may be analyzed and/or validated by a mobile app.

In some embodiments, the kit comprises a sterile swab. After taking a nasal (anterior nares) or cheek swab sample, the swab is inserted into a sample tube and mixed. The swab is removed and a lysis cap is added to the sample tube. The lysis cap comprises an UDG (thermolabile Uracil DNA glycosylase) lyophilized bead, which is exposed to the solution as the sample tube is inverted. The tube is heated, and the lysis cap is removed and is replaced by an amplification cap. The amplification cap comprises a reverse transcriptase and RPA lyophilized bead. The sample tube, now comprising the amplification cap, is then inverted until the bead dissolves. Then, the sample tube is heated, and the sample tube is added to a readout device (and a dilution buffer is added). The readout device then runs the same through a lateral flow test, and the results of the test (e.g., positive or negative for the viral illness(es) screened) are reported in a mobile app. The readout device comprises a clear window so that the user can view the lateral flow strip and the test results. In some embodiments, the readout device further comprises markings near the window so that the companion mobile app is able to register and acquire an image in order to process the results.

In further embodiments, the user takes a picture of the lateral flow strip with their smartphone that is running the mobile application. The lateral flow strip is clearly visible through the transparent viewing window in the readout device. The readout device contains markers that allow the mobile app to recognize the proper orientation of the image and provide feedback to the user. After uploading the image, a computer vision algorithm is run to electronically call the bands. If the band-pattern result determined by the algorithm differs from the band pattern result entered by the user, the user is asked to double-check that they entered the correct band-pattern, and the user is given the opportunity to redo to the “Record Results” page. Alternatively, in some embodiments, the interpretation is performed solely by the computer-vision algorithm. Based on the result that the user entered, the user is shown the corresponding “Test Complete” screen in the mobile application, which tells the user if the test result is positive, negative, or invalid. In addition to providing the test result, careful language is used to ensure that the user can properly interpret the meaning of the result.

In another embodiment, the kit comprises a sterile swab, a cap, an amplification cap, a heating device, and a readout device. After taking a nasal or cheek swab sample, the swab is inserted into a sample tube. The swab is removed and a cap is added to the sample tube. The tube is then placed in the heating device and heated. The cap is removed and is replaced by an amplification cap. Then, the tube is heated and then added to a readout device, and the readout device then runs the same through a lateral flow test, and the results of the test (e.g., positive or negative for the viral illness(es) screened) using ARUCO markers, are reported in a mobile app.

In another embodiment, the kit comprises a sterile swab, a blister cap, a heating device, and a readout device. After taking a nasal or cheek swab sample, the swab is inserted into a sample tube. The swab is removed and the blister cap is added to the sample tube. The tube is then placed in the heating device and heated. The blister cap is then pushed, so that it releases its cargo, in this case, an amplification pellet comprising the lyophilized reagents necessary for amplification of the sample. The sample tube, now comprising the amplification cap, is then inverted until the bead dissolves. Then, the sample tube is heated and then the sample tube is added to a readout device, and the readout device then runs the same through a lateral flow test, and the results of the test (e.g., positive or negative for the viral illness(es) screened) are determined with ARUCO markers, and are reported in a mobile app.

In another embodiment, the kit comprises a tube comprising UDG reagents, a cap comprising amplification reagents, a heating device, and a readout device. The user takes a sample and adds the sample to the tube. A cap is applied to the tube and then the tube is placed in the heating device for UDG treatment (to prevent potential cross-contamination) and lysis. When heating is complete, the user then removes the tube from the heating device. The user removes the cap from the tube and replaces it with the cap comprising amplification reagents (e.g., LAMP-associated reagents or RPA-associated reagents). The user then shakes the tube briefly to mix the components, and then places it back in the heating device. After heating is complete, the user removes the tube from the heating device and runs the sample through a readout device (e.g., a lateral flow test designed to screen for COVID-19 and influenza). In some embodiments, the results of the test are interpreted and/or provided by a companion mobile application described herein.

In some embodiments, a diagnostic test kit comprises a heating unit. The heating unit may be any device capable of heating fluidic contents of a reaction tube. In certain embodiments, the heating unit is a battery-powered heat source, a USB-powered heat source, a hot plate, a heating coil, or a hot water bath. In some embodiments, the heating unit is contained within a thermally-insulated housing to ensure user safety. In some embodiments, the heating unit is an off-the-shelf consumer-grade device.

1. A test ecosystem configured to process a test reading or a test result of a rapid test for COVID-19 and/or an influenza virus and/or a target nucleic acid, wherein the rapid test is an isothermal nucleic acid amplification-based test.

2. The test ecosystem of claim 1, wherein the test ecosystem comprises a computing resource configured to store the test reading or the test result.

3. The test ecosystem of claim 2, wherein the computing resource is a central computing resource, a clinician computing resource, a medical record computing resource, and/or an agency computing resource.

4. The test ecosystem of claim 3, wherein the test ecosystem is configured to integrate the test reading or the test result with subject data.

5. The test ecosystem of claim 4, wherein the subject data is account data, tracking data, test record data, and/or clinical data.

6. The test ecosystem of claim 5, wherein the test ecosystem is configured to perform contact tracing based on the tracking data.

7. The test ecosystem of claim 5, wherein the test record data comprises antibody test data, COVID-19 test data, influenza test data, and/or target nucleic acid test data.

8. The test ecosystem of claim 4, wherein the testing ecosystem is configured to access at least a first portion of the subject data from the clinician computing resource, the medical record computing resource, and/or the agency computing resource.

9. The test ecosystem of claim 4, wherein the test ecosystem is configured to store the test reading, the test result, and/or the subject data in the central computing resource.

10. The test ecosystem of claim 9, wherein the testing ecosystem is configured to transmit the test reading, the test result, and/or at least a second portion of the subject data to the clinician computing resource, the medical record computing resource, and/or the agency computing resource.

11. The test ecosystem of claim 1, wherein the test reading or the test result may be entered manually by a user.

12. The test ecosystem of claim 1, wherein the test reading or the test result is a test reading, and the test reading may be entered by a user through uploading an image of the test reading using a downloadable software application.

13. The test ecosystem of claim 12, wherein the test ecosystem determines the test result automatically from the entered test reading.

14. An apparatus comprising:
at least one computer hardware processor; and
at least one non-transitory computer-readable storage medium storing processor executable instructions that, when executed by the at least one computer hardware processor, cause the at least one computer hardware processor to perform:
processing a reading of a rapid test for COVID-19 and/or an influenza virus and/or a target nucleic acid, wherein the rapid test is an isothermal nucleic acid amplification-based test.

15. The apparatus of claim 14, further comprising a computing resource configured to store the reading.

16. The apparatus of claim 15, wherein the computing resource is a central computing resource, a clinician computing resource, a medical record computing resource, and/or an agency computing resource.

17. The apparatus of claim 14, wherein the instructions are configured to cause the at least one computer hardware processor to integrate the test reading or the test result with subject data.

18. The apparatus of claim 17, wherein the subject data is account data, tracking data, test record data, and/or clinical data.

19. The apparatus of claim 18, wherein the instructions are configured to cause the at least one processor to perform contact tracing based on the tracking data.

20. A non-transitory computer-readable media comprising instructions that, when executed by one or more processors on a computing device, are operable to cause the one or more processors to:
process a reading of a rapid test for COVID-19 and/or an influenza virus and/or a target nucleic acid, wherein the rapid test is an isothermal nucleic acid amplification-based test.
